SEQUENCE LISTING (48 sequences)

SEQ ID NO: 1 - protein, 60 aa, Artificial Sequence
  synthetic
  LIPID (1)..(1): MYRISTATE
  MOD_RES (60)..(60): AMIDATION
GTNLSVPNPLGFFPDHQLDPAFGANSNNPDWDFNPNKDHWPEANQVGAGAFGPGFTPPHG

SEQ ID NO: 2 - protein, 55 aa, Artificial Sequence
  synthetic
  LIPID (1)..(1): MYRISTATE
  MOD_RES (55)..(55): AMIDATION
GTNLSVPNPLGFFPDHQLDPAFGANSNNPDWDFNPNKDHWPEANQVGAGAFGPGF

SEQ ID NO: 3 - protein, 47 aa, Artificial Sequence
  synthetic
  LIPID (1)..(1): MYRISTATE
  MOD_RES (47)..(47): AMIDATION
GTNLSVPNPLGFFPDHQLDPAFGANSNNPDWDFNPNKDHWPEANQVG

SEQ ID NO: 4 - protein, 40 aa, Artificial Sequence
  synthetic
  LIPID (1)..(1): MYRISTATE
  MOD_RES (40)..(40): AMIDATION
GTNLSVPNPLGFFPDHQLDPAFGANSNNPDWDFNPNKDHW

SEQ ID NO: 5 - protein, 35 aa, Artificial Sequence
  synthetic
  LIPID (1)..(1): MYRISTATE
  MOD_RES (35)..(35): AMIDATION
GTNLSVPNPLGFFPDHQLDPAFGANSNNPDWDFNP

SEQ ID NO: 6 - protein, 30 aa, Artificial Sequence
  synthetic
  LIPID (1)..(1): MYRISTATE
  MOD_RES (30)..(30): AMIDATION
GTNLSVPNPLGFFPDHQLDPAFGANSNNPD

SEQ ID NO: 7 - protein, 25 aa, Artificial Sequence
  synthetic
  LIPID (1)..(1): MYRISTATE
  MOD_RES (25)..(25): AMIDATION
GTNLSVPNPLGFFPDHQLDPAFGAN

SEQ ID NO: 8 - protein, 20 aa, Artificial Sequence
  synthetic
  LIPID (1)..(1): MYRISTATE
  MOD_RES (20)..(20): AMIDATION
GTNLSVPNPLGFFPDHQLDP

SEQ ID NO: 9 - protein, 58 aa, Artificial Sequence
  synthetic
  LIPID (1)..(1): MYRISTATE
  MOD_RES (58)..(58): AMIDATION
GGWSSKPRQGMGTNLSVPNPLGFFPDHQLDPAFGANSNNPDWDFNPNKDHWPEANQVG

SEQ ID NO: 10 - protein, 57 aa, Artificial Sequence
  synthetic
  LIPID (1)..(1): MYRISTATE
  MOD_RES (57)..(57): AMIDATION
GLSWTVPLEWGTNLSVPNPLGFFPDHQLDPAFGANSNNPDWDFNPNKDHWPEANQVG

SEQ ID NO: 11 - protein, 47 aa, Artificial Sequence; identical to SEQ ID NO: 3
  synthetic
  LIPID (1)..(1): PALMITATE
  MOD_RES (47)..(47): AMIDATION

SEQ ID NO: 12 - protein, 47 aa, Artificial Sequence; identical to SEQ ID NO: 3
  synthetic
  MOD_RES (1)..(1): ACETYLATION - N-term Stearoyl
  MOD_RES (47)..(47): AMIDATION

SEQ ID NO: 13 - protein, 47 aa, Artificial Sequence; identical to SEQ ID NO: 3
  synthetic
  MOD_RES (1)..(1): ACETYLATION - N-term Chol
  MOD_RES (47)..(47): AMIDATION

SEQ ID NO: 14 - protein, 47 aa, Artificial Sequence
  synthetic
  LIPID (1)..(1): MYRISTATE
  MOD_RES (47)..(47): AMIDATION
GTNLSVPNPLGFFPDHQLDPAFGANSNNPDWDFNPVKDDWPAANQVG

SEQ ID NO: 15 - protein, 47 aa, Artificial Sequence
  synthetic
  LIPID (1)..(1): MYRISTATE
  MOD_RES (47)..(47): AMIDATION
GTNLSVPNPLGFFPDHQLDPAFKANSENPDWDLNPNKDNWPDANKVG

SEQ ID NO: 16 - protein, 47 aa, Artificial Sequence
  synthetic
  LIPID (1)..(1): MYRISTATE
  MOD_RES (47)..(47): AMIDATION
GQNLSTSNPLGFFPDHQLDPAFRANTANPDWDFNPNKDTWPDANKVG

SEQ ID NO: 17 - protein, 47 aa, Artificial Sequence
  synthetic
  LIPID (1)..(1): MYRISTATE
  MOD_RES (47)..(47): AMIDATION
GKNISTTNPLGFFPDHQLDPAFRANTRNPDWDHNPNKDHWTEANKVG

SEQ ID NO: 18 - protein, 47 aa, Artificial Sequence
  synthetic
  LIPID (1)..(1): MYRISTATE
  MOD_RES (47)..(47): AMIDATION
GQNLSVPNPLGFFPDHQLDPLFRANSSSPDWDFNTNKDSWPMANKVG

SEQ ID NO: 19 - protein, 47 aa, Artificial Sequence
  synthetic
  LIPID (1)..(1): MYRISTATE
  MOD_RES (47)..(47): AMIDATION
GKNLSASNPLGFLPDHQLDPAFRANTNNPDWDFNPKKDPWPEANKVG

SEQ ID NO: 20 - protein, 47 aa, Artificial Sequence
  synthetic
  LIPID (1)..(1): MYRISTATE
  MOD_RES (47)..(47): AMIDATION
GQNLSVPNPLGFFPDHQLDPLFRANSSSPDWDFNTNKDNWPMANKVG

SEQ ID NO: 21 - protein, 60 aa, Hepatitis B virus; identical to SEQ ID NO: 1

SEQ ID NO: 22 - protein, 55 aa, Hepatitis B virus; identical to SEQ ID NO: 2

SEQ ID NO: 23 - protein, 47 aa, Hepatitis B virus; identical to SEQ ID NO: 3

SEQ ID NO: 24 - protein, 40 aa, Hepatitis B virus; identical to SEQ ID NO: 4

SEQ ID NO: 25 - protein, 35 aa, Hepatitis B virus; identical to SEQ ID NO: 5

SEQ ID NO: 26 - protein, 30 aa, Hepatitis B virus; identical to SEQ ID NO: 6

SEQ ID NO: 27 - protein, 25 aa, Hepatitis B virus
GTNLSVPNPLGFFPDHQLDPAFGAN

SEQ ID NO: 28 - protein, 20 aa, Hepatitis B virus
GTNLSVPNPLGFFPDHQLDP

SEQ ID NO: 29 - protein, 58 aa, Hepatitis B virus; identical to SEQ ID NO: 9

SEQ ID NO: 30 - protein, 57 aa, Hepatitis B virus; identical to SEQ ID NO: 10

SEQ ID NO: 31 - protein, 47 aa, Hepatitis B virus; identical to SEQ ID NO: 3

SEQ ID NO: 32 - protein, 47 aa, Hepatitis B virus; identical to SEQ ID NO: 3

SEQ ID NO: 33 - protein, 47 aa, Hepatitis B virus; identical to SEQ ID NO: 3

SEQ ID NO: 34 - protein, 47 aa, Hepatitis B virus; identical to SEQ ID NO: 14

SEQ ID NO: 35 - protein, 47 aa, Hepatitis B virus; identical to SEQ ID NO: 15

SEQ ID NO: 36 - protein, 47 aa, Hepatitis B virus; identical to SEQ ID NO: 16

SEQ ID NO: 37 - protein, 47 aa, Hepatitis B virus; identical to SEQ ID NO: 17

SEQ ID NO: 38 - protein, 47 aa, Hepatitis B virus; identical to SEQ ID NO: 18

SEQ ID NO: 39 - protein, 47 aa, Hepatitis B virus; identical to SEQ ID NO: 19

SEQ ID NO: 40 - protein, 47 aa, Hepatitis B virus; identical to SEQ ID NO: 20

SEQ ID NO: 41 - DNA, 3221 bp, Hepatitis B virus
ttccactgccttccaccaagctctgcaggatcccaaagtcaggggtctgtattttcctgctggtggctccagttcaggaacagtcaaccctgctccaaatattgcctctcacatctcgtcaatctccgcgaggactggggaccctgtgccgaacatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctatggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaaaccctaacaaaacaaaaagatggggttattccctaaacttcatggtctacataattggaagttggggaacgttgccacaggatcatattgtacaaaagatcaaacactgttttagaaaacttcctgttaacagacctattgattggaaagtatgtcaaagaattgtgggtcttttgggctttgctgctccatttactcaatgtggatatcctgccttaatgcctttgtatgcatgtatacaagctaaacaggcttttactttctcgccaacttacaaggcctttctaagtaaacagtatatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcatcggaactgataattctgtcgtcctctcgcggaaatatacatcgtttccatggctgctaggctgtactgccaactggatccttcgcgggacgtcctttgtttacgtcccgtcggcgctgaatcccgcggacgacccctcgcggggccgcttgggagtctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactctcagcaatgtcaatgaccgaccttgaggcctacttcaaagactgtgtgtttaaggactgggaggagcttggggaggagattaggttaaaggtgtttgtattaggaggctgtaggcataaattggtctgcgcaccagcaccatgcaactttttcacctctgcctaatcatctcttgtacatgtcccacttttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctgagcattgctctcctcaccatactgcactcaggcaagccattctctgctgggtggaattgatgactctagccacctgggtgggtaataatttggaagatccagcatccagggatctagtagtcaattatgttaatactaacatgggtttaaagatcaggcaactattgtggtttcatatatcttgtcttacttttggaagacagactgtgctagaatatttggtctctttcggagtgtggattcgcactcctccagcttatagaccaccaaatgcccctatcttatcaacacttccggagactactgttgttagacgacgggaccgaggcaggtcccctagaagaagaactccctcgcctcgcagacgcagatctcaatcgccgcgtcgcagaagatctcaatctcgggaatctcaatgttagtattccttggactcataaggtgggaaattttactgggctttattcctctacagtacctatctttaatcctgaatggcaaactccttcctttcctaagattcatttacaagaggacattattaataggtgtcaacaatttgtgggccctctcactgtaaatgaaaagagaagactgaaattaattatgcctgccagattctatcctacccacactaaatatttgcccttagacaaaggaattaaaccttattatccagatcaggtagttaatcattacttccaaaccagacattatttacatactctttggaaggctgggattctatataagagggaaaccacacgtagcgcatcattttgcgggtcaccatattcttgggaacaagagctacatcatgggaggttggtcatcaaaacctcgcaaaggcatggggacgaacctttctgttcccaaccctctgggattctttcccgatcatcagttggaccctgcattcggagccaattcaaacaatccagattgggacttcaaccccatcaaggaccactggccagcaggcaaccaggtaggagtgggagcattcgggccaaggctcacccctccacacggcggtattttggggtggagccctcaggctcagggcatattggccacagtgtcaacaattcctcctcctgcctccaccaatcggcagtcaggaaggcagcctactcccatctctccacctctaagagacagtcatcctgaggccatgcagtggaa

SEQ ID NO: 42 - DNA, 3215 bp, Hepatitis B virus
aactccaccactttccaccaaactcttcaagatcccagagtcagggccctgtaccttcctgctggtggctccagttcagaaatagtgagccctgctcagaatactgtctctgccatatcgtcaatcttatcgaagactggggaccctgtaccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacctgttgtcctccaatttgtcctgggtatcgctggatgtgtctgcggcgttttatcatattcctctgcatcctgctgctatgcctcatcttcttgttggttcttctggactatcaaggtatgttgcccgtttgtcctctaattccaggatcatcaacaaccagcaccggaccatgcaaaacctgcacaactcctgctcaaggaacctctatgtttccctcatgttgctgtacaaaacctacggacggaaactgcacctgtattcccatcccatcatcttgggctttcgcaaaattcctatgggagtgggcctcagtccgtttctcttggctcagtttactagtgccatttgttcagtggttcgtagggctttcccccactgtctggctttcagttatatcgatgatgtggttttgggggccaagtctgtacaacatcttgagtccctttataccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaaatgtgttttaagaaacttcctgtaaacaggcctattgattggaaagtatgtcaacgaattgtgggtcttttggggtttgccgccccttttacgcaatgtggatatcctgctttaatgcctttatatgcatgtatacaagcaaaacaggcttttactttctcgccaacttacaaggcctttctaagtaaacagtatatgaacctttaccccgttgctcggcaacggcctggtctgtgccaagtgtttgctgacgcaacccccactggttggggcttggccattggccatcagcgcatgcgtgggacctttgtgtctcctctgccgatccatactgcggaactcctagccgcttgttttgctcgcagcaggtctggggcaaaactcatcgggactgacaattctgtcgtgctctcccgcaagtatacatcatttccatggctgctaggctgtgctgccaactggatcctgcgcgggacgtcctttgtttacgtcccgtcggcgctgaatcccgcggacgacccctcccggggccgcttggggctctaccgcccgcttctccgcctgttgtaccgaccgaccacggggcgcacctctctttacgcggactccccgtctgtgccttctcatctgccggaccgtgtgcacttcgcttcacctctgcacgtcgcatggagaccaccgtgaacgcccacaggaacctgcccaaggtcttgcataagaggactcttggactttcagcaatgtcaacgaccgaccttgaggcatacttcaaagactgtgtgtttactgagtgggaggagttgggggaggagattaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgcggagttactctcttttttaccttctgacttctttccttctattcgagatcttctcgacaccgcctctgctctgtatcgggaggccttagagtctccggaacattgttcacctcaccatacggcactcaggcaagctattctgtgttggggtgagttgatgaatctagccacctgggtgggaagtaatttggaagatccagcatccagggaattagtagtcggctatgtcaacgttaatatgggcctaaaacttagacaactattgtggtttcacatttcctgtcttacttttgggagagaaactgttcttgaatatttggtgtcttttggagtgtggattcgcactccttctgcatatagaccaccaaatgcccctatcctatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgttttaatcctaaatggcaaactccttcttttcctgacattcatttgcaggaggacattgttgatagatgtaagcaatttgtggggccccttacagtaaatgaaaacaggagactaaaattaattatgcctgctaggttttatcccaatgttactaaatatttgcccttagataaagggatcaaaccgtattatccagagtatgtagttaatcattacttccagacgcgacattatttacacactctttggaaagcggggatcttatataaaagagagtccactcgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatggggggctgttggggtggagccctcaggctcagggcctactcacaactgtgccagcagctcctcctcctgcctccaccaatcggcagtcaggaaggcagcctactcccttatctccacctctaagggacactcatcctcaggccatgaagtgg

SEQ ID NO: 43 - DNA, 3215 bp, Hepatitis B virus
aattccacaacattccaccaagctctgctagaccccagagtgaggggcctatactttcctgctggtggctccagttccggaacagtaaaccctgttccgactactgcctcacccatatcgtcaatcttctcgaggactggggaccctgcaccgaacatggagagcacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgagtcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccatgtctgtacaacatcttgagtccctttttacctctattaccaattttcttttgtctttgggtatacatttgaaccctaataaaaccaaacgttggggctactcccttaacttcatgggatatgtaattggaagttggggtactttaccgcaagaacatattgtacaaaaacttaagcaatgttttcgaaaactgcctgtaaatagacctattgattggaaagtatgtcagagaattgtgggtcttttgggctttgctgccccttttacacaatgtggctatcctgctttaatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggctattggccatcgccgcatgcgtggaacctttgtggctcctctgccgatccatactgcggaactcctagcagcttgttttgctcgcagccggtctggagcaaaacttatcggcaccgacaactctgttgtcctctctcggaagtacacctcctttccatggctgctagggtgtgctgccaactggatcctgcgcgggacgtcctttgtttacgtcccgtcggcgctgaatcccgcggacgacccgtctcggggccgtttgggactctaccgtccccttcttcatctgccgttccggccgaccacggggcgcacctctctttacgcggtctccccgtctgtgccttctcatctgccggaccgtgtgcacttcgcttcacctctgcacgtcgcatggagaccaccgtgaacgcccaccaggtcttgcccaaggtcttacataagaggactcttggactctcagcaatgtcaacgaccgaccttgaggcatacttcaaagactgtttgtttaaggactgggaggagttgggggaggagattaggttaaaggtctttgtactaggaggctgtaggcataaattggtctgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcgcctcaccatacagcactcaggcaagctattctttgttggggtgagttgatgaatctggccacctgggtgggaagtaatttggaagacccagcatccagggaattagtagccagctatgtcaatgttaatatgggcctaaaaatcagacaactactgtggtttcacatttcctgtcttacttttggaagagaaactgttcttgagtatttggtatcttttggagtgtggattcgcactcctcctgcttacagaccaccaaatgcccctatcttatcaacacttccggaaactactgttgttagacgacgaggcaggtcccctagaagacgaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatccagagtggcaaactccctcctttcctcacattcatttacaggaggacattattaatagatgtcaacaatatgtgggccctcttacagttaatgaaaaaagaagattaaaattaattatgcctgctaggttctatcctaaccttaccaaatatttgcccttggacaaaggcattaaaccatattatcctgaacatgcagttaatcattacttcaaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacaaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcagggcatactgacaacagtgccagtagcacctcctcctgcctccaccaatcggcagtcaggaagacagcctactcccatctctccacctctaagagacagtcatcctcaggccatgcagtgg

SEQ ID NO: 44 - DNA, 3182 bp, Hepatitis B virus
aactccacaaccttccaccaaactctgcaagatcccagagtgagaggcctgtatttccctgctggtggctccagttcaggaacagtaaaccctgttccgactactgtctctcccatatcgtcaatcttctcgaggattggggaccctgcgctgaacatggagaacatcacatcaggattcctaggacccctgctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcttcaactaccagcacgggaccatgcagaacctgcacgactcctgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtcgggctttcccccactgtttggctttcagttatatggatgatgtggtattgggggccaagtctgtacagcatcttgagtccctttttaccgctgttaccaattttcttttgtctttgggtatacatttaaaccctgacaaaacaaaaagatggggttactctttacatttcatgggctatgtcattggaagttatgggtcattgccacaagatcacatcatacaaaaaatcaaagaatgttttcgaaaacttcctgttaacagacctattgattggaaagtctgtcaacgtattgtgggtcttttgggttttgctgccccttttacacaatgtggttatcctgctttaaagcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaacctttctggctcctctgccgatccatactgcggaactcctagccgcttgttttgctcgcagcaggtctggagcaaacattctcgggacggataactctgttgttctctcccgcaaatatacatcgtttccatggctgctaggctgtgctgccaactggatcctgcgcgggacgtcctttgtttacgtcccgtcggcgctgaatcccgcggacgacccttctcggggtcgcttgggactctctcgtccccttctccgtctgccgtttcgaccgaccacggggcgcacctctctttacgcggactccccgtctgtgccttctcatctgccggaccgtgtgcacttcgcttcacctctgcccgtcgcatggagaccaccgtgaacgcccaccgagtcttgcccaaggtcttacataagaggactcttggactctctgtaatgtcaacgaccgaccttgaggcatacttcaaagactgtttgttcaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttagggcatggacatcgatccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcagttctgtgctggggggaactaatgactctagctacctgggtgggtggtaatttggaagatccaacatccagggacctagtagtcagttatgtcaacactaatatgggcctaaagttccggcaactattgtggtttcacatttcttgtctcacttttggaagagaaacagttttagagtatttggtgtctttcggagtgtggattcgcactcctccagcttatagaccaccaaatgcccctatcttatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtgggaaactttacggggctttattcttctactgtacctgtctttaaccctcattggaaaacaccctcttttcctaatatacatttacaccaagacattatcaaaaaatgtgaacaatttgtaggcccactcacagtcaatgaaaaaagaagactgcaattgattatgcctgctaggttttatccaaatgttaccaaatatttgccattggataagggtatcaaaccttattacccagaacatctagttaatcattacttccaaaccagacattatttacacactctatggaaggcgggtatattatataagagagaaacaacacatagcgcctcattttgtgggtcaccatattcttgggaacaaaagctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggattcaccccaccgcacggaggccttttggggtggagccctcaggctcagggcatactacaaaccttgccagcaaatccgcctcctgcctctacccatcgccagtcaggaaggcagcctaccccgctgtctccacctttgagaaacactcatcctcaggccatgcagtgg

SEQ ID NO: 45 - DNA, 3212 bp, Hepatitis B virus
ttccacagcattccaccaagctctgcaggatcccaaagtaagaggcctgtattttcctgctggtggctccagttccggaacagtgagccctgttccgactactgcctcactcatctcgtcaatcttctcgaggattggggaccctgcaccgaacatggaaggcatcacatcaggattcctgggacccctgctcgtgttacaggcggggtttttcttgttgacaaaaatcctcacaataccgcagagtctagactcgtggtggacttctctcaattttctagggggagctcccgtgtgtcttggccaaaattcgcagtccccaacctccaatcactcaccaacctcttgtcctccgatttgtcctggctatcgctggatgtgtctgcggcgttttatcatcttcctcttcatcctgctgctatgcctcatcttcttgttggttcttctggactatcaaggtatgttgcccgtttgtcctctaattccaggatcatcaaccaccagtacgggaccctgccgaacctgcacgactcttgctcaaggaacctctatgtttccctcatgttgctgttcaaaaccttcggacggaaattgcacttgtattcccatcccatcatcatgggctttcggaaaattcctatgggagtgggcctcagcccgtttctcctggctcagtttactagtgccatttgttcagtggttcgccgggctttcccccactgtctggctttcagttatatggatgatgtggtattgggggccaagtctgtacaacatcttgagtccctttatacctctgttaccaattttcttttgtctttgggtatacatttaaatcccaacaaaacaaaacgatggggatattctcttaatttcatgggatatgtaattgggagttgggggtcattaccacaggaacacatccgaatgaaaatcaaagactgttttagaaaactccctgttaaccggcctattgattggaaagtatgtcaaagaattgtgggtcttttgggctttgctgccccttttacacaatgtggatatcctgctttaatgcctctgtatgcgtgtgttcaatcgaagcaggctttcactttctcgccaacttacaaggcctttctgtgtaaacaatacctgaacctttaccccgttgcccggcaacggccaggtctgtgccaagtgtttgctgatgcaacccccactggctggggcttggccataggccatcagcgcatgcgtggaacctttgtggctcctctgccgatccatactgcggaactcctagccgcttgttttgctcgcagcaggtctggagcgaaacttatcgggacggataattctgtcgttctctcccggaaatatacatcatttccatggctgctaggctgtgctgccaactggatcctgcgagggacgtcctttgtctacgtcccgtcagcgctgaatcctgcggacgacccgtctcggggtcgcttggggatctatcgtccccttctccgtctgccgttccggccgaccacggggcgcacctctctttacgcggtctccccgtctgtgccttctcatctgccggaccgtgtgcacttcgcttcacctctgcacgtcgcatggagaccaccgtgaacgcccatcaaatcttgcccaaggtcttacataagcggactcttggactttctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacattgacccttataaagaatttggagctactgtggagttactctcgtttttgccgtctgacttctttccgtcagttagagatcttctagataccgcctcagctctgtatcgggatgccttagaatctcctgagcattgttcacctcaccatactgcactcaggcaagccattctttgctggggggaactaatgactctagctacctgggtgggtgtaaatttggaagatccagcatccagggacctagtagtcagttatgtcaatactaatatgggcctaaagttcaggcaattattgtggtttcacatttcttgtctcacttttggaagagaaaccgtcatagagtatttggtgtcttttggagtgtggattcgcactcctccagcttatagaccaccaaatgcccctatcttatcaacacttccggagaatactgttgttagacgaagaggcaggtcccctagaagaagaactccctcgcctcgcagacgaagatctcaatcgccgcgtcgcagaagatctcaatctccagcttcccaatgttagtattccttggactcacaaggtgggaaattttacgggcctttactcttctactatacctgtctttaatcctaactggaaaactccatcttttcctgatattcatttgcaccaagacattattaacaaatgtgaacaatttgtaggtcctctaacagtaaatgaaaaacgaagattaaacttagtcatgcctgctagattttttcccatctctacgaaatatttgcccctagagaagggtataaaaccttattatccagataatgtagttaatcattacttccaaaccagacactatttacataccctatggaaggcgggcatcttatataaacgggaaactacacgtagcgcctcattttgtgggtcaccttattcttgggaacaagagctacatcatggggctttcttggacggtccctctcgaatgggggaagaatcattccaccaccaatcctctgggattttttcccgaccaccagttggatccagcattcagagcaaacaccagaaatccagattgggaccacaatcccgacaaagaccactggacagaagccaacaaggtaggagtgggagcattcgggccggggttcactcccccacacggaggccttttggggtggagccctcaggctcaaggcatgctaaaaacattgccagcagatccgcctcctgcctccaccaatcggcagtcaggaaggcagcctaccccaatcactccacctttgagagacactcatcctcaggccatgcagtggaa

SEQ ID NO: 46 - DNA, 3215 bp, Hepatitis B virus
ctccactcagttccaccaggctctgttagatccgagagtaagggctctgtattttcctgctggtggctccagttcagagacacagaaccctgctccgactattgcctctctcacatcatcaatcttcttgaagactgggggccctgctacgaacatggacaacatcacatcaggactcctaggacccctgctcgtgttacaggcggtgtgtttcttgttgacaaaaatcctcacaataccacagagtctagactcgtggtggacttctctcaattttctagggggaacacccgggtgtcctggccaaaattcgcagtccccaacctccaatcacttaccaacctcctgtcctccaacttgtcctggctatcgctggatgtgtctgcggcgttttatcatcttcctcttcatcctgctgctatgcctcatcttcttgttggttcttctggactatcaaggtatgttgcccgtttgtcctctacttccaggatccacgaccaccagcacgggaccatgcaaaacctgcacaactcttgctcaaggaacctctatgtttccctcttgctgctgttccaaaccctcggacggaaactgcacttgtattcccatcccatcatcctgggctttaggaaaatacctatgggagtgggcctcagcccgtttctcctggctcagtttactagtgcaatttgttcagtggtgcgtagggctttcccccactgtctggcttttagttatatggatgatctggtattgggggccaaatctgtgcagcatcttgagtccctttataccgctgttaccaattttctgttatctgtgggtatccatttaaatacctcgaaaacaaaaagatggggttataccctaaatttcatgggttatgttattggcagttggggatcattaccacaagatcacattgtacaaaaaatcaaagattgttttcgtaaacttcctgtaaatcgccctattgattggaaagtttgtcaacgcattgtgggtcttttgggctttgccgccccctttactcaatgtggttatcctgctctcatgcctctgtatgcctgtataactgctaaacaggcttttgtcttttcgccaacttacaaggcctttctatgtcaacaatacatgaacctttaccccgttgctcggcaacggccaggcctgtgccaagtgtttgctgacgcaacccccactggttggggcttggccattggccatcagcgcatgcgtggaacctttgtggctcctctgccgatccatactgcggaactccttgcagcttgtttcgctcgcagccggtctggagcgaaactcatcggcacagacaactctgttgtcctctctaggaagtacacctccttcccatggctgctcggttgtgctgccaactggatcctacgcgggacgtcctttgtttacgtcccgtcggcgctgaatccagcggacgatccctctcggggtcgcttggggctgtatcgcccccttctccgtctgccgttccagccgacgacgggtcgcacctctctttacgcggcctccccgtctgttccttctcgtctgccggaccgtgtgcacttcgcttcacctctgcacgtcgcatggagaccaccgtgaacgcccctcgaagcttgccaacagtcttacataagcggactcttggactttcaggaaggtcaatcacctggatcgaagaatacatcaaagactgtgtatttaaggactgggaggagctgggggaggagattaggttaaaggtctttgtattaggaggctgtaggcataaattggtctgttcaccagcaccatgcaactttttcacctctgcctaatcatcttttgttcatgtcctactgttcaagcctccaagctgtgccttgggtggctttggggcatggacattgacccttataaagaatttggagcttctgtggaattactctcttttttgccttctgacttcttcccgtcagttcgggacctactcgacaccgcttcagccctctaccgggatgctttagaatcaccagaacattgcacacctaaccataccgctctcaggcaagctatattgtgctggggtgagttaatgactttggcttcctgggtgggcaataacttggaagatcctgctgctagggacctagtggttaactatgtcaatactaacatgggcctaaaaattagacaattgctgtggtttcacatttcctgccttacttttggaagagaaacagttcttgagtatttggtgtcttttggagtgtggattcgcactcctcctgcttatagaccaccaaatgcccctatcttatccacacttccggaaactactgttgttagacgacgaggcaggtcccctcgaagaagaactccctcgcctcgcagacgaaggtctcaatcgccgcgtcgcagaagatctcaatctccagcttcccaatgttagtattccttggactcataaggtgggaaattttacgggactctattcctctactgttcctacttttaatcctgactggttaactccttcttttcctgacattcatttacatcaagatttgatacacaaatgtgaacaatttgtaggccctctcacaaaaaatgaattgagaagattaaaattagttatgccatccagattttttcctaaggttaccaaatattttcctatggaaaagggaattaaaccctattatcctgataacgtggttaatcattattttaagaccagacactatttgcatactttatggaargcrggcattctatataagagagaatccacacgtagcgcctcattttgtgggtcaccatattcttgggaacaagagctacagcatgggagcacctctatcaacgactcgaaggggcatgggacagaatctctctgtacccaatcctctgggattctttccagaccatcagctggatcctctattcagggcaaattccagcagtcccgactgggacttcaacaaaaacaaggacaattggccaatggcaaacaaggtaggagtgggaggatacggtcctgggttcacacccccacacggtggcctgttggggtggagtccacaggcacagggtgtgcttacaacattgccagcagatccgcctcctgcttccaccaatcggcggtccgggagaaaaccaaccccagtctctccacctctaagagacactcatccacaggccatgcagtggaa

SEQ ID NO: 47 - DNA, 3248 bp, Hepatitis B virus
ctctacagcattccaccaagctctacaaaatcccaaagtcaggggcctgtattttcctgctggtggctccagttcagggatagtgaaccctgttccgactattgcctctcacatctcgtcaatcttctccaggattggggaccctgcaccgaacatggagaacatcacatcaggattcctaggacccctgctcgtgttacaggcggggtttttcttgttgacaagaatcctcacaataccgcagagtctagactcgtggtggacttctctcaattttctagggggagtgcccgtgtgtcctggcctaaattcgcagtccccaacctccaatcactcaccaatctcctgtcctccaacttgtcctggctatcgctggatgtgtctgcggcgttttatcatattcctcttcatcctgctgctatgcctcatcttcttgttggttcttctggactatcaaggtatgttgcccgtttgtcctctgattccaggatcctcgaccaccagcacgggaccctgcaaaacctgcacgactcctgctcaaggcaactctatgtatccctcatgttgctgtacaaaaccttcggacggaaattgcacctgtattcccatcccatcatcttgggctttcgcaaaatacctatgggagtgggcctcagtccgtttctcttggctcagtttactagtgccatttgttcagtggttcgtagggctttcccccactgtctggctttcagctatatggatgatgtggtattgggggccaaatctgtacaacatcttgagtccctttataccgctgttaccaattttcttttgtctttgggtatacatctaaaccctaacaaaacaaaaagatggggttattccttaaattttatgggatatgtaattggaagttggggtactttgccacaagaacacatcacacagaaaattaagcaatgttttcggaaactccctgttaacaggccaattgattggaaagtctgtcaacgaataactggtctgttgggtttcgctgctccttttacccaatgtggttaccctgccttaatgcctttatatgcatgtatacaagctaagcaggcttttactttctcgccaacttataaggcctttctctgtaaacaatacatgaacctttaccccgttgctaggcaacggcccggtctgtgccaagtgtttgctgacgcaacccccactggttggggcttggccatcggccatcagcgcatgcgtggaacctttgtggctcctctgccgatccatactgcggaactcctagctgcttgttttgctcgcagccggtctggagcaaaactcattgggactgacaattctgtcgtcctttctcggaaatatacatcctttccatggctgctaggctgtgctgccaactggatccttcgcgggacgtcctttgtttacgtcccgtcagcgctgaatccagcggacgacccctcccggggccgtttggggctctgtcgcccccttctccgtctgccgttcctgccgaccacggggcgcacctctctttacgcggtctccccgtctgttccttctcatctgccggaccgtgtgcacttcgcttcacctctgcacgttacatggaaaccgccatgaacacctctcatcatctaccaaggcagttatataagaggactcttggactgtttgttatgtcaacaaccgggatggagaaatacttcaaggactgtgtttttgctgagtgggaagaattaggcaatgagtccaggttaatgacctttgtattaggaggctgtaggcataaattggtctgcgcaccagcaccatgtaactttttcacctctgcctaatcatctcttgttcatgtcctactgttcaagcctccaagctgtgccttgggtggctttagggcatggatagaacaactttgccatatggcctttttggcttagacattgacccttataaagaatttggagctactgtggagttgctctcgtttttgccttctgactttttcccgtctgttcgtgatcttctcgacaccgcttcagctttgtaccgggaatccttagagtcctctgatcattgttcgcctcaccatacagcactcaggcaagcaattctgtgctggggtgagttgatgactctagctacctgggtgggtaataatttggaagatccagcatccagagatttggtggtcaattatgttaatactaatatgggtttaaaaatcaggcaactattgtggtttcacatttcctgtcttacttttgggagagaaaccgttcttgagtatttggtgtcttttggagtgtggattcgcactcctcctgcttatagaccaccaaatgcccctatcctatcaacacttccggagactactgttgttagacgaagaggcaggtcccctcgaagaagaactccctcgcctcgcagacgaagatctcaatcgccgcgtcgcagaagatctgcatctccagcttcccaatgttagtattccttggactcacaaggtgggaaactttacggggctgtattcttctactatacctgtctttaatcctgattggcaaactccttcttttccaaatatccatttgcatcaagacattataactaaatgtgaacaatttgtgggccctctcacagtaaatgagaaacgaagattaaaactagttatgcctgccagatttttcccaaactctactaaatatttaccattagacaaaggtatcaaaccgtattatccagaaaatgtagttaatcattacttccagaccagacattatttacataccctttggaaggcgggtattctatataagagagaaacatcccgtagcgcttcattttgtgggtcaccatatacttgggaacaagatctacagcatggggctttcttggacggtccctctcgagtggggaaagaacctttccaccagcaatcctctaggattccttcccgatcaccagttggacccagcattcagagcaaataccaacaatccagattgggacttcaatcccaaaaaggacccttggccagaggccaacaaggtaggagttggagcctatggacccgggttcacccctccacacggaggccttttggggtggagccttcagtctcagggcacactaacaactttgccagcagatccgcctcctgcctccaccaatcgtcagtcagggaggcagcctactcccatctctccaccactaagagacagtcatcctcaggccatgcagtggaa

SEQ ID NO: 48 - DNA, 3215 bp, Hepatitis B virus
ctcaacacagttccaccaagcactgttggatccgagagtaagggggctgtattttcctgctggtggctccagttcagaaacacagaaccctgctccgactattgcctctctcacatcatcaatcttctcgaagactggggaccctgctatgaacatggagaacatcacatcaggactcctaggaccccttctcgtgttacaggcggtgtgtttcttgttgacaaaaatcctcacaataccaaagagtctagactcgtggtggacttctctcaattttctaggggtaccacccgggtgtcctggccaaaattcgcagtccccaatctccaatcacttaccaacctcctgtcctccaacttgtcctggctatcgttggatgtgtctgcggcgttttatcatcttcctcttcatcctgctgctatgcctcatcttcttgttggttcttctggactatcaaggtatgttgcccgtgtgtcctctacttccaggatcwacaaccaccagcacgggaccctgcaaaacctgcaccactcttgctcaaggaacctctatgtttccctcctgctgctgtaccaaaccttcggacggaaattgcacctgtattcccatcccatcatcttgggctttcggaaaatacctatgggagtgggcctcagcccgtttctcttggctcagtttactagtgcaatttgttcagtggtgcgtagggctttcccccactgtctggcttttagttatatggatgatctggtattgggggccaaatctgtgcagcatcttgagtccctttataccgctgttaccaattttttgttatctgtgggcatccatttgaacacagctaaaacaaaatggtggggttattcccttcactttatgggttatataattgggagttgggggaccttgcctcaggaacatattgtgcataaaatcaaagattgctttcgcaaacttcccgtgaatagacccattgattggaaggtttgtcaacgcattgtgggtcttttgggctttgcagccccttttactcaatgtggttatcctgctctcatgcccttgtatgcctgtattrccgctaagcaggcttttgttttctcgccaacttacaaggcctttctctgtaaacaatacatgaacctttaccccgttgctcggcaacggccaggcctttgccaagtgtttgctgacgcaacccccactggctggggcttggcgattggccatcagcgcatgcgcggaacctttgtggctcctctgcccatccatactgcggaactcctagccgcttgtttcgctcgcagcaggtctggagcggacattatcggcactgacaactccgttgtcctttctcggaagtacacctccttcccatggctgctaggctgtgctgccaactggatcctgcgcgggacgtcctttgtctacgtcccgtcggcgctgaatcctgcggacgacccctctcgtggtcgcttggggctctgccgccctcttctccgcctgccgttccggccgacgacgggtcgcacctctctttacgcggactccccgcctgtgccttctcatctgccggcccgtgtgcacttcgcttcacctctgcacgtcgcatggagaccaccgtgaacgccccttggaacttgccaacaaccttacataagaggactcttggactttcgccccggtcaacgacctggattgaggaatacatcaaagactgtgtatttaaggactgggaggagtcgggggaggagttgaggttaaaggtctttgtattaggaggctgtaggcataaattggtctgttcaccagcaccatgcaactttttcacctctgcctaatcatcttttgttcatgtcccactgttcaagcctccaagctgtgccttgggtggctttggggcatggacattgacccttataaagaatttggagcttctgtggagttactctcatttttgccttctgacttcttcccgtctgtccgggacctactcgacaccgcttcagccctctaccgagatgccttagaatcaccagaacattgcacccccaaccacactgctctcaggcaagctattttgtgctggggtgagttgatgaccttggcttcctgggtgggcaataatttagaggatcctgcagcaagagatctagtagttaattatgtcaatactaacatgggcctaaaaattagacaattattatggtttcacatttcttgccttacatttggaagagaaactgtgcttgagtatttggtgtcttttggagtgtggattcgcactccacctgcttatagaccaccaaatgcccctatcctatcaacacttccggagactactgttgttagacaacgaggcagggcccctagaagaagaactccctcgcctcgcagacgaagatctcaatctccgcgtcgcagaagatctcaatctccagcttcccaatgttagtattccttggactcataaggtgggaaactttaccggtctttactcctctactatacctgttttcaatcctgactggttaactccttcttttcctgacattcacttgcatcaagatctgatacaaaaatgtgaacaatttgtaggcccactcactacaaatgaaaggagacgattgaaactaattatgccagctaggttttatcccaaagttactaaatacttccctttggataaaggtattaagccttactatccagagaatgtggttaatcattactttaaaactagacattatttacatactttgtggaaggcaggaattctatataagagagaatccacacatagcgcctcattttgtgggtcaccatattcctgggaacaagagctacagcatgggagcacctctctcaacggcgagaaggggcatgggacagaatctttctgtgcccaatcctctgggattctttccagaccaccagttggatccactattcagagcaaattccagcagtcccgattgggacttcaacacaaacaaggacaattggccaatggcaaacaaggtaggagtgggaggcttcggtccagggttcacacccccacacggtggccttctggggtggagccctcaggcacagggcattctaacaacctcgccaccagatccacctcctgcttccaccaatcggaggtcaggaagaaagccaaccccagtctctccacctctaagggacacacatccacaggccatgcagtggaa

(19) United States
(12) Patent Application Publication
(10) Pub. No.: US 2020/0338158 A1
(43) Pub. Date: Oct. 29, 2020

(54) Therapeutic Drug for Non-Alcoholic Fatty Liver Disease

(71) Applicant: Shanghai Hep Pharmaceutical Co., Ltd., Shanghai (CN)

(72) Inventor: Hongli LIU, Shanghai (CN)

(21) Appl. No.: 16/757,067

(22) PCT Filed: Oct. 18, 2018
(86) PCT No.: PCT/CN2018/110770

§ 371 Date: Apr. 17, 2020

(30) Foreign Application Priority Data
Oct. 18, 2017 (CN) 201710972548.6

(51) Int. Cl. A61K 38/16; A61P 1/16

(52) U.S. Cl. CPC A61K 38/162; A61P 1/16

(57) ABSTRACT

The disclosure relates to therapeutic medicaments for nonalcoholic fatty liver disease. Specifically, the disclosure relates to use of a polypeptide comprising an amino acid sequence derived from hepatitis B virus (HBV) or a pharmaceutical composition comprising the polypeptide in the manufacture of a medicament for treating or preventing nonalcoholic fatty liver disease.

### TECHNICAL FIELD

This disclosure relates to therapeutic medicaments for non-alcoholic fatty liver disease.

### BACKGROUND

Nonalcoholic fatty liver disease (NAFLD) is a clinical pathological disease, which has liver histological changes similar to alcoholic fatty liver disease, but does not involve an excessive drinking history; and NAFLD belongs to an acquired metabolic stress liver injury that is closely related to insulin resistance and genetic susceptibility, and its pathology includes simple fatty liver (SFL), nonalcoholic steatohepatitis (NASH), and fatty liver fibrosis and cirrhosis, as the disease develops.

Insulin resistance is closely related to the risk of nonalcoholic fatty liver. Recent studies have shown that almost all patients with nonalcoholic fatty liver disease have insulin resistance in liver and peripheral tissues, which is not necessarily accompanied by abnormal glucose tolerance or obesity. It is shown that the severity of insulin resistance is associated with the development of non-alcoholic fatty liver disease. Foreign studies have shown that, by upregulating colony growth factor, insulin plays a key role in the pathogenesis of nonalcoholic fatty liver.

Lipid metabolism disorder is common in patients having nonalcoholic fatty liver. Studies have shown that about 50% of the patients with lipid metabolism disorder have fatty liver. The incidence rate of fatty liver in patients with severe hypertriglyceridemia and mixed hyperlipidemia is 5-6 times higher than that in healthy subjects.

Almost all NASH patients are more than 10% heavier than healthy subjects, and about ⅓ of NASH patients have type II diabetes. Most NASH patients do not have obvious symptoms, but some of them feel fatigue or malaise continuously or intermittently, and a few of them have dull pain in the right upper abdomen, all of which are nonspecific. When NASH develops into cirrhosis and liver failure showing their corresponding symptoms and disorder, it is difficult at this time to distinguish them from cirrhosis and liver failure caused by other causes.

Nonalcoholic fatty liver disease can directly lead to decompensated cirrhosis, hepatocellular carcinoma and liver transplantation recurrence, and also affect the development of other chronic liver diseases, and further involve in the pathogenesis of type II diabetes and atherosclerosis. Metabolic syndrome-related malignancies, atherosclerotic cardiovascular and cerebrovascular diseases and cirrhosis affect life quality and life expectancy of patients with nonalcoholic fatty liver disease. Therefore, treatment of nonalcoholic fatty liver disease is challenging in the field of medicine.

### SUMMARY

This disclosure provides compositions and methods for treating nonalcoholic fatty liver disease (NAFLD) with a polypeptide derived from HBV. In some embodiments, the polypeptides described herein include polypeptides derived from the pre-S1 region of any one of HBV genotypes A, B, C, D, E, F, G, and H.

In some aspects, the disclosure provides a pharmaceutical composition comprising a polypeptide described herein, wherein when administered to a subject in need thereof, the pharmaceutical composition is capable of treating nonalcoholic fatty liver disease (NAFLD) of the subject.

In some aspects, the disclosure provides methods of treating nonalcoholic fatty liver disease (NAFLD) in a subject in need thereof, comprising administering to the subject a therapeutically effective amount of the polypeptide described herein or a pharmaceutical composition comprising the polypeptide.

In some aspects, the nonalcoholic fatty liver disease (NAFLD) described herein comprises simple fatty liver (SFL), nonalcoholic steatohepatitis (NASH), fatty liver fibrosis and cirrhosis.

In some embodiments, the polypeptide described herein comprises an amino acid sequence derived from the pre-S1 region of HBV genotype A, B, C, D, E, F, G, or H. In certain embodiments, the polypeptide described herein comprises the sequence of amino acids 13-59 of the pre-S1 region of HBV genotype C. In additional embodiments, the polypeptide described herein comprises an amino acid sequence derived from the pre-S1 region of any other HBV genotype that corresponds to amino acids 13-59 of the pre-S1 region of HBV genotype C. In some embodiments, the polypeptide comprises an amino acid sequence selected from SEQ ID NOs: 21-40.

In some embodiments, one or more amino acid residues of the polypeptide described herein are deleted, substituted, or inserted while maintaining the biological activity described herein. In certain embodiments, the polypeptide described herein comprises a native flanking amino acid sequence from the pre-S1 region of HBV genotype A, B, C, D, E, F, G, or H. In other embodiments, the polypeptide described herein has at least about 30%, 40%, 50%, 60%, 70%, 80%, 85%, 90%, 95%, 96%, 97%, 98%, or 99% identity to any one of the amino acid sequences selected from SEQ ID NOs: 21-40. In some embodiments, the polypeptide comprises the glycine corresponding to amino acid 13 of the pre-S1 region of HBV genotype C and/or the asparagine corresponding to amino acid 20 of the pre-S1 region of HBV genotype C.

In some embodiments, the polypeptide described herein comprises an N-terminal modification with a hydrophobic group and/or a C-terminal modification that is capable of stabilizing the polypeptide. The hydrophobic group may be chosen from, e.g., myristic acid, palmitic acid, stearic acid, oleic acid, linoleic acid, cholesterol, and arachidonic acid. The C-terminal modification may be chosen from, e.g., amidation (amination), isopentanediolization, and any C-terminal modification that is capable of stabilizing the polypeptide. In certain embodiments, the polypeptide described herein comprises an N-terminal modification with myristic acid and/or a C-terminal modification with amination. In some embodiments, the polypeptide described herein comprises an amino acid sequence chosen from SEQ ID NOs: 21-40. In some embodiments, the polypeptide described herein comprises the amino acid sequence of SEQ ID NO: 23.

In one aspect, the polypeptide described herein is capable of reducing one or more symptoms associated with nonalcoholic fatty liver disease. In some embodiments, the polypeptide described herein or the pharmaceutical composition comprising such polypeptide is administered to the subject before, concurrently with, or after the administration of a therapeutically effective amount of at least one a second agent. The second agent may be chosen from, e.g., an antihyperlipidemic agent, an antihyperglycemic agent, an antidiabetic agent, an antiobesity agent, and a bile acid analogue. For example, the second agent may be chosen from, e.g., insulin, metformin, sitagliptin, colesevelam, glipizide, simvastatin, atorvastatin, ezetimibe, fenofibrate, nicotinic acid, orlistat, lorcaserin, phentermine, topiramate, obeticholic acid, and ursodeoxycholic acid.

In some embodiments, the polypeptide described herein or the pharmaceutical composition comprising such polypeptide is administered to the subject by at least one mode including, e.g., parenteral, intrapulmonary, intranasal, intralesional, intramuscular, intravenous, intraarterial, intraperitoneal, and subcutaneous administration. In some embodiments, the polypeptide described herein or the pharmaceutical composition comprising such polypeptide is administered to the subject subcutaneously.

### DESCRIPTION OF DRAWINGS

FIG. 1 shows comparison of liver weight of mice at week 8.

FIG. 2 shows comparison of liver weight of mice at week 16.

FIG. 3 shows CT quantitative result of body fat distribution in vivo of mice at week 16.

FIG. 4 shows HE staining of mouse livers at week 8 (40×200).

FIG. 5 shows HE staining of mouse livers at week 16 (40×200).

FIG. 6 shows oil red 0 staining of mouse livers at week 8 (40×200).

FIG. 7 shows oil red 0 staining of mouse livers at week 16 (40×200).

FIG. 8 shows Masson staining of mouse livers at week 8 (40×200).

FIG. 9 shows Masson staining of mouse livers at week 16 (40×200).

FIG. 10 shows TUNEL staining of mouse livers at week 8 (400×200).

FIG. 11 shows TUNEL staining of mouse livers at week 16 (400×200).

FIG. 12 shows semi-quantitative results of liver cell apoptosis for mice at week 8 (40×200, for 10 fields).

FIG. 13 shows semi-quantitative results of liver cell apoptosis for mice at week 16 (40×200, for 10 fields).

FIG. 14 shows GTT test result in mice at week 8.

FIG. 15 shows GTT test result in mice at week 16.

FIG. 16 shows serum insulin level in mice at week 16.

FIG. 17 shows HOMA-IR result in mice at week 16.

FIG. 18 shows serum ALB, ALP level in mice at week 16.

FIG. 19 shows serum HDL-C, LDL-C level in mice at week 8.

FIG. 20 shows serum HDL-C, LCL-C level in mice at week 16.

FIG. 21 shows serum VLDL level in mice at week 16.

FIG. 22 shows serum ALT, AST level in mice at week 8.

FIG. 23 shows serum ALT, AST level in mice at week 16.

FIG. 24 shows serum TG, TC level in mice at week 8.

FIG. 25 shows serum TG, TC level in mice at week 16.

FIG. 26 shows liver TG level in mice at week 8.

FIG. 27 shows liver TG level in mice at week 16.

FIG. 28 shows liver TC level in mice at week 8.

FIG. 29 shows liver TC level in mice at week 16.

FIG. 30 shows liver hydroxyproline level in mice at week 8.

FIG. 31 shows liver hydroxyproline level in mice at week 16.

FIG. 32 shows liver FFA level in mice at week 8.

FIG. 33 shows liver FFA level in mice at week 16.

### DETAILED DESCRIPTION

It should be appreciated that, any combination of the technical features described above and those to be described in detail below (e.g. in the Examples) can be used within the scope of the disclosure to form preferred technical solutions.

Unless defined otherwise, all technical and scientific terms used herein have the same meaning as commonly understood by those of ordinary skill in the art to which the disclosure belongs. For the purposes of the present disclosure, the following terms are defined below.

The articles “a” and “an” refer to one or to more than one (i.e., to at least one) of the grammatical object of the article. For example, “an element” means one element or more than one element.

The term “or” means, and is used interchangeably with, the term “and/or,” unless context clearly indicates otherwise.

To the extent that the term “contain,” “include,” “have,” or grammatical variants of such term are used in either the disclosure or the claims, such term is inclusive in a manner similar to the term “comprising” as “comprising” is interpreted when employed as a transitional word in a claim. The term “including” or its grammatical variants mean, and are used interchangeably with, the phrase “including but not limited to.”

The term “about” means a quantity, level, value, number, frequency, percentage, dimension, size, amount, weight, or length that varies by as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth. In general, the term “about” is intended to modify a numerical value above and below the stated value by a variance of ≤10%.

I. Polypeptides

Certain aspects of the present disclosure provide polypeptides derived from HBV for treating nonalcoholic fatty liver disease in a subject. The polypeptides may be derived from the pre-S1 region of HBV. The subject may be a mammal. In some embodiments, the subject may be a human.

The terms “polypeptide,” “peptide,” and “protein” are used interchangeably and encompass full-length proteins and fragments, as well as variants of the full-length proteins and the fragments. Such fragments and variants of the polypeptide described herein retain at least the biological activities of hepalatide. The “polypeptide,” “peptide,” and “protein” can include natural and/or non-natural amino acid residues. Those terms also include post-translationally modified proteins, including, e.g., glycosylated, sialylated, acetylated, and/or phosphorylated proteins. The terms also include chemically modified proteins at one or more amino acid residues, such as, e.g., at the N-terminus and/or at the C-terminus. For instance, the N-terminus of the polypeptide disclosed herein can be modified by a hydrophobic group such as, e.g., myristic acid, palmitic acid, stearic acid, oleic acid, linoleic acid, cholesterol, and arachidonic acid. In some embodiments, the C-terminus of the polypeptide disclosed herein can be modified to stabilize the polypeptide. The C-terminus modification may be chosen from amidation (amination), isopentanediolization, and any other C-terminal modification capable of stabilizing the polypeptide.

As used herein, the term “polypeptide derived from HBV” or “HBV-derived polypeptide” refers to the origin or source of the polypeptide as being from HBV, and may include native, recombinant, synthesized, or purified polypeptides. The term “polypeptide derived from HBV” or “HBV-derived polypeptide” refers to a full-length native HBV polypeptide or fragments thereof, as well as variants of the full-length native polypeptide or its fragments. In some embodiments, the fragment may consist of at least 3-5 amino acids, at least 5-10 amino acids, at least 10-20 amino acids, at least 20-30 amino acids, at least 30-50 amino acids, or the entire amino acids of the native sequence, or may be otherwise identifiable to one of ordinary skill in the art as having its origin in the native sequence. In some embodiments, the polypeptide described herein may be derived from the pre-S1 region of the L protein of any HBV subtype. In some embodiment, the polypeptide described herein may comprise the entire pre-S1 region of the L protein of any HBV subtype. In certain embodiments, the polypeptide described herein may be derived from the pre-S1 region of the L protein of any one of HBV genotypes A, B, C, D, E, F, G, and H. The genomic sequences of these HBV genotypes can be found in GenBank Accession Nos. KC875260 (SEQ ID NO: 41), AY220704 (SEQ ID NO: 42), AF461363 (SEQ ID NO: 43), AY796030 (SEQ ID NO: 44), AB205129 (SEQ ID NO: 45), DQ823095 (SEQ ID NO: 46), HE981176 (SEQ ID NO: 47), and AB179747 (SEQ ID NO: 48), respectively. In certain embodiments, the polypeptide described herein may be derived from the pre-S1 region of the L protein of HBV genotype C. The polypeptide derived from HBV described herein retains one or more biological activities described herein of the corresponding native HBV polypeptide.

“Variant” as used herein in connection with the polypeptide described herein, a polypeptide derived from HBV, or an HBV-derived polypeptide means a polypeptide that differs from a given polypeptide (i.e., the polypeptide described herein, the polypeptide derived from HBV, or the HBV-derived polypeptide) in amino acid sequence, but retains one or more biological activities described herein of the given polypeptide. The variant polypeptide described herein may have one or more amino acid additions (e.g., insertion), deletions, or substitutions from the given polypeptide. In some embodiments, the variant polypeptide described herein may have 1-30, 1-20, 1-10, 1-8, 1-5, or 1-3 amino acid additions (e.g., insertion), deletions, or substitutions from the given polypeptide, including all integers in between these ranges. For example, the polypeptide sequence may contain conservative substitution of amino acids. A conservative substitution of an amino acid, i.e., replacing an amino acid with a different amino acid of similar properties (e.g., hydrophilicity and degree and distribution of charged regions), typically involves a minor change and therefore does not significantly alter the biological activity of the polypeptide. These minor changes can be identified, in part, by considering the hydropathic index of amino acids based on a consideration of the hydrophobicity and charge of the amino acid. Amino acids of similar hydropathic indexes and hydrophilicity values can be substituted and still retain protein function. Both the hydrophobicity index and the hydrophilicity value of amino acids are influenced by the particular side chain of that amino acid. Consistent with that observation, amino acid substitutions that are compatible with biological function depend on the relative similarity of the amino acids, and particularly the side chains of those amino acids, as revealed by the hydrophobicity, hydrophilicity, charge, size, and other properties.

The term “variant” also includes a polypeptide that has certain identity, such as, e.g., at least about 30%, 40%, 50%, 60%, 70%, 80%, 85%, 90%, 95%, 96%, 97%, 98%, or 99% identity to the given polypeptide. “Variant” as used herein also includes a polypeptide comprising the portion of the given polypeptide that corresponds to a native sequence of HBV proteins. “Variant” may also refer to a fusion protein or chimeric protein, comprising polypeptides derived from two or more different sources. Non-limiting examples of the fusion protein described herein may include, e.g., a fusion protein of one polypeptide derived from HBV and another polypeptide derived from a non-HBV protein, a fusion protein of two polypeptides derived from different HBV subtypes, and a fusion protein of two polypeptides derived from different regions of the L protein of any one of HBV subtypes, or from different sequences within the pre-S1 region of the L protein of any one of HBV subtypes.

The term “variant” also includes a polypeptide that comprises the same amino acid sequence of a given polypeptide (i.e., the polypeptide described herein, the polypeptide derived from HBV, or the HBV-derived polypeptide) and retains one or more biological activities of the given polypeptide, but chemically and/or post-translationally modified in a manner different from the given polypeptide. “Variant” can also be used to describe a polypeptide or a fragment thereof that has been differentially processed, such as by proteolysis, phosphorylation, or other post-translational modification, yet retains its biological activity of binding to NTCP and bidirectionally regulating NTCP-mediated transport of bile acid into hepatocytes. Use of “variant” herein is intended to encompass fragments of a variant unless otherwise contradicted by context. The term “variant” also encompasses the homologous polypeptide sequences found in the different viral species, strains, or subtypes of the hepadnavirus genus. HBV is divided into four major serotypes (adr, adw, ayr, ayw) based on antigenic epitopes present on its envelope proteins, and into eight genotypes (A-H) according to overall nucleotide sequence variation of the genome. The term “variant” therefore includes homologous polypeptides found in any of these HBV subtypes. “Variant” can also include polypeptides having native flanking amino acid sequences from any of these HBV subtypes added to the N and/or C terminus.

The terms “conservative amino acid substitutions” and “conservative substitutions” are used interchangeably herein to refer to intended amino acid swaps within a group of amino acids wherein an amino acid is exchanged with a different amino acid of similar size, structure, charge, and/or polarity. Families of amino acid residues having similar side chains are known in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in some embodiments, an amino acid residue in a polypeptide can be replaced with another amino acid residue from the same side chain family. In other embodiments, a string of amino acids can be replaced with a structurally similar string that differs in order and/or composition of side chain family members. In yet other embodiments, mutations can be introduced randomly along all or part of the polypeptide. Examples of conservative amino acid substitutions include, e.g., exchange of one of the aliphatic or hydrophobic amino acids Ala, Val, Leu, and Ile for one of the other amino acids in that group of four; exchange between the hydroxyl-containing residues Ser and Thr; exchange between the acidic residues Asp and Glu; exchange between the amide residues Asn and Gln; exchange between the basic residues Lys, Arg, and His; exchange between the aromatic residues Phe, Tyr, and Trp; and exchange between the small-sized amino acids Ala, Ser, Thr, Met, and Gly. Conservative substitutions, such as substituting a conserved amino acid with a similar, structurally related amino acid would not be reasonably expected to impose a substantial influence on the biological activity of the polypeptide.

The term “sequence identity” (e.g., a “sequence 50% identical to”) refers to the extent that a sequence is identical on an amino acid-by-amino acid basis over a window of comparison. In some embodiments, the polypeptide described herein may comprise an amino acid sequence at least about 30%, 40%, 50%, 60%, 70%, 80%, 85%, 90%, 95%, 96%, 97%, 98%, or 99% identical to the sequence of a given polypeptide and still retain one or more biological activities of the given polypeptide. A “percentage identity” (or “% identity”) may be calculated by comparing two optimally aligned sequences over the window of comparison, determining the number of positions at which the identical amino acids occur in both sequences to yield the number of matched positions, dividing the number of matched positions by the total number of positions in the window of comparison, and multiplying the result by 100 to yield the percentage of sequence identity. Optimal alignment of sequences for aligning a comparison window may be conducted by computerized implementations of algorithms available in the art, such as, e.g., the BLAST® family of programs, or by visual inspection and the best alignment (i.e., resulting in the highest percentage homology over the comparison window) generated by any of the various methods selected. For sequence comparison, one sequence acts as a reference sequence, to which test sequences are compared. When using a sequence comparison algorithm, test and reference sequences are input into a computer, subsequence coordinates may be designed, if necessary, and sequence algorithm program parameters are designated. The sequence comparison algorithm then calculates the present sequence identity for the test sequences relative to the reference sequence, based on the designated program parameters. The designation of sequence algorithm program parameters is well within the knowledge in the art. For example, the window of comparison may be designated as over the entire length of either or both comparison sequences, such as, e.g., over the entire length of the reference sequence, and gaps of up to 5% of the total number of amino acids in the reference sequence may be allowed.

As used herein, the “biological activity” of the polypeptides described herein encompasses the ability of the polypeptides to decrease deposition of subcutaneous fat, liver fat and serum fat in a subject (such as C57BL6 mice), decrease serum ALT and/or AST level, improve apoptosis and decrease liver fibrosis. In some aspects, the “biological activity” described herein may also encompass the ability to increase total bile acid (TBA) level and decrease blood albumin (ALB), HDL and LDL level in a subject. In some aspects, the “biological activity” described herein may also include the ability to decrease blood glucose and insulin level and improve insulin resistance in a subject.

In certain embodiments, the “biological activity” of the polypeptide described herein includes the ability of the polypeptide to ameliorate or prevent one or more symptoms or complications of such disorders. In certain embodiments, the “biological activity” of the polypeptide described herein includes the ability of the polypeptide to mitigate the negative impact of such disorders on the health of a patient or reduce the risk of developing such disorders. In certain embodiments, the “biological activity” of the polypeptide described herein also includes the ability of the polypeptide to reduce the severity of or the risk of developing other associated diseases, such as, e.g., atherosclerosis and/or cardiovascular diseases, heart diseases, kidney impairment, or obesity.

Various in vivo, in vitro, and ex vivo assays to confirm the biological activity of the polypeptide described herein are contemplated. The biological activity of the polypeptide described herein may be confirmed in vivo, by collecting a sample from a subject treated with the polypeptide described herein. The sample may be a biopsy sample collected from a specific tissue such as, e.g., liver, muscle, fat, and pancreas, or a snap-frozen tissue collected from an animal post-mortem. In some embodiments, the sample may be a serum sample collected from blood drawn from a subject. Various methods for collecting a serum sample from a subject are known in the art, and include, e.g., tail-bleeding, retro-orbital puncture, and cardiopuncture. In some embodiments, the biological activity of the polypeptide described herein may be confirmed in vitro, by contacting the polypeptide described herein with a cell that is either a transformed cell line or a cell isolated from an animal. In some embodiments, the cell may be a primary hepatocyte isolated from an animal.

The exemplary assays useful to confirm the biological activity of the polypeptide may also include a functional analysis with a sample collected from a subject treated with the polypeptide described herein, including, e.g., glucose production assay, glucose uptake assay, fatty acid oxidation assay, cholesterol assay, bile acids assay, urea assay, and triglyceride assay.

In some embodiments, the polypeptide described herein may comprise an amino acid sequence of the pre-S1 region of any HBV subtype. In some embodiments, the polypeptide described herein comprises the sequence of amino acids 13-59 of the pre-S1 region of HBV genotype C: GTNLSVPNPLGFFPDHQLDPAFGANSNNPDWDFNPNKDHWPEANQVG (SEQ ID NO: 23). In additional embodiments, the polypeptide described herein may comprise the corresponding pre-S1 sequence from another HBV genotype, such as, e.g., any one of genotypes A, B, D, E, F, G, and H. For example, in some embodiments, the polypeptide described herein may comprise:

pre-S1 amino acids 13-59 of HBV genotype A:
(SEQ ID NO: 34)
GTNLSVPNPLGFFPDHQLDPAFGANSNNPDWDFNPVKDDWPAANQVG,
pre-S1 amino acids 13-59 of HBV genotype B:
(SEQ ID NO: 35)
GTNLSVPNPLGFFPDHQLDPAFKANSENPDWDLNPNKDNWPDANKVG,
pre-S1 amino acids 2-48 of the HBV genotype D:
(SEQ ID NO: 36)
GQNLSTSNPLGFFPDHQLDPAFRANTANPDWDFNPNKDTWPDANKVG,
pre-S1 amino acids 12-58 of the HBV genotype E:
(SEQ ID NO: 37)
GKNISTTNPLGFFPDHQLDPAFRANTRNPDWDHNPNKDHWTEANKVG,
pre-S1 amino acids 13-59 of the HBV genotype F:
(SEQ ID NO: 38)
GQNLSVPNPLGFFPDHQLDPLFRANSSSPDWDFNTNKDSWPMANKVG,
pre-S1 amino acids 12-58 of the HBV genotype G:
(SEQ ID NO: 39)
GKNLSASNPLGFLPDHQLDPAFRANTNNPDWDFNPKKDPWPEANKVG,
or
pre-S1 amino acids 13-59 of the HBV genotype H:
(SEQ ID NO: 40)
GQNLSVPNPLGFFPDHQLDPLFRANSSSPDWDFNTNKDNWPMANKVG.

In some embodiments, the polypeptide described herein may comprise a portion of the pre-S1 region of HBV, said portion comprising at least an amino acid sequence chosen from SEQ ID NOs: 23 and 34-40. In some embodiments, the polypeptide described herein may comprise the entire pre-51 region of HBV.

In some embodiments, the polypeptide described herein may be 10-100 amino acids in length. For example, the polypeptide may be 15-100, 15-80, 20-100, 20-80, 20-60, 25-60, 30-60, 35-60, or 40-60 amino acids in length, including all integers in between these ranges. In some embodiments, the polypeptide described herein may be at least 20, such as, e.g., at least 25, 30, 35, 40, amino acids in length. In some embodiments, the polypeptide described herein may be 20, 25, 30, 35, 40, 47, 55, 60 amino acids in length. In some embodiments, the polypeptide described herein may be 47 amino acids in length. The variants of the polypeptides described herein that differ in length retain one or more biological activities associated with the corresponding polypeptides.

In some embodiments, the polypeptide described herein may comprise an N-terminal modification with a hydrophobic group. For example, the hydrophobic group may be chosen from myristic acid, palmitic acid, stearic acid, oleic acid, linoleic acid, cholesterol, and arachidonic acid. In some embodiments, the hydrophobic group may be chosen from myristic acid, palmitic acid, stearic acid, and cholesterol. In some embodiments, the hydrophobic group may be myristic acid. In certain embodiments, the polypeptide described herein may comprise an amino acid sequence chosen from SEQ ID NOs: 23 and 34-40, wherein the N terminus may be modified with a hydrophobic group chosen from myristic acid, palmitic acid, stearic acid, and cholesterol. In certain embodiments, the polypeptide described herein may comprise an amino acid sequence chosen from SEQ ID NOs: 23 and 34-40, wherein the N terminus may be myristoylated. In some embodiments, the polypeptide described herein may comprise the amino acid sequence of SEQ ID NO: 23, wherein the N terminus may be myristoylated. In some embodiments, the polypeptide described herein may comprise a C-terminal modification to stabilize the polypeptide. For example, the C-terminal modification may be chosen from amidation (amination), isopentanediolization, and any other C-terminal modification capable of stabilizing the polypeptide described herein. In some embodiments, the C-terminal modification may be amidation (amination). For example, the polypeptide described herein may comprise the amino acid sequence of NO: 23, wherein the N terminus may be myristoylated, and/or the C terminus may be amidated (aminated). In some embodiments, the polypeptide described herein may comprise the amino acid sequence of NO: 3 (Cmyr-47). In some embodiments, the polypeptide described herein may comprise an amino acid sequence chosen from SEQ ID NOs: 34-40, wherein the N terminus may be myristoylated, and/or the C terminus may be modified by amidated (aminated). In some embodiments, the polypeptide described herein may comprise an amino acid sequence chosen from SEQ ID NOs: 14-20. The variants of the polypeptide described herein that are modified at the N-terminus and/or the C-terminus retain one or more biological activities of the corresponding polypeptides that are not modified in the same manner, including at least the biological activity of binding to NTCP and bidirectionally regulating NTCP-mediated transport of bile acid into hepatocytes.

Variants of the polypeptides described herein are also contemplated in the present disclosure, including variants with one or more amino acid deletions, substitutions, or insertions that retain one or more biological activities of the polypeptides. The polypeptides described herein preferably retain the glycine corresponding to amino acid 13 of the pre-S1 region of HBV genotype C (i.e., the N-terminal glycine of SEQ ID NO: 23). In some embodiments, the polypeptides described herein retain the asparagine corresponding to amino acid 20 of the pre-S1 region of HBV genotype C. In some embodiments, the polypeptide described herein may have one or more naturally-occurring mutations in the pre-S1 region of HBV. In some embodiments, the polypeptide described herein may have 1-30, such as, e.g., 1-20, 1-10, 1-8, 1-5, or 1-3, amino acid deletions, substitutions, or insertions relative to a sequence from the pre-S1 region of HBV, including all integers in between these ranges. In some embodiments, the polypeptide described herein may have 1-30, such as, e.g., 1-20, 1-10, 1-8, 1-5, or 1-3, amino acid deletions, substitutions, or insertions relative to an amino acid sequence chosen from SEQ ID NOs: 23 and 34-40, including all integers in between these ranges. In some embodiments, the polypeptide described herein may have 1-30, such as, e.g., 1-20, 1-10, 1-8, 1-5, or 1-3, amino acid deletions, substitutions, or insertions relative to the amino acid sequence of SEQ ID NO: 23, including all integers in between these ranges. In some embodiments, the polypeptide described herein may have 1-3 amino acid deletions, substitutions, or insertions from the amino acid sequence of SEQ ID NO: 23. In certain embodiments, the polypeptide described herein may have 1-30, such as, e.g., 1-20, 1-10, 1-8, 1-5, or 1-3, amino acid deletions or insertions at the C terminus of an amino acid sequence chosen from SEQ ID NOs: 23 and 34-40, including all integers in between these ranges. For example, the polypeptide described herein may comprise an amino acid sequence chosen from SEQ ID NOs: 21, 22, and 24-28. In some embodiments, the polypeptide described herein may comprise the amino acid sequence of any one of the polypeptides listed in Table 1. In some embodiments, the polypeptide described herein may be chosen from any one of the post-translationally modified polypeptides listed in Table 1.

TABLE 1
List of Exemplary Polypeptides

| SEQ | | N-terminal | Amino acid sequence | C-terminal | |
|---|---|---|---|---|---|
| ID | SEQ | Modifi- | 123456789012345678901234567890 | Modifi- | |
| No. | name | cation | 12 | cation | SEQ origin |
| 1 | Cmyr-60 | Myr | GTNLSVPNPLGFFPDHQLDPAFGANSNNPD | NH2 | Genotype C Pre-S1(13-72) |
| | | | WDFNPNKDHWPEANQVGAGAFGPGFTPPHG | | |
| 2 | Cmyr-55 | Myr | GTNLSVPNPLGFFPDHQLDPAFGANSNNPD | NH2 | Genotype C Pre-S1(13-67) |
| | | | WDFNPNKDHWPEANQVGAGAFGPGF | | |
| 3 | Cmyr-47 | Myr | GTNLSVPNPLGFFPDHQLDPAFGANSNNPD | NH2 | Genotype C Pre-S1(13-59) |
| | | | WDFNPNKDHWPEANQVG | | |
| 4 | Cmyr-40 | Myr | GTNLSVPNPLGFFPDHQLDPAFGANSNNPD | NH2 | Genotype C Pre-S1(13-52) |
| | | | WDFNPNKDHW | | |
| 5 | Cmyr-35 | Myr | GTNLSVPNPLGFFPDHQLDPAFGANSNNPD | NH2 | Genotype C Pre-S1(13-47) |
| | | | WDFNP | | |
| 6 | Cmyr-30 | Myr | GTNLSVPNPLGFFPDHQLDPAFGANSNNPD | NH2 | Genotype C Pre-S1(13-42) |
| 7 | Cmyr-25 | Myr | GTNLSVPNPLGFFPDHQLDPAFGAN | NH2 | Genotype C Pre-S1(13-37) |
| 8 | Cmyr-20 | Myr | GTNLSVPNPLGFFPDHQLDP | NH2 | Genotype C Pre-S1(13-32) |
| 9 | Cmyr-47 + | Myr | GGWSSKPRQGMGTNLSVPNPLGFFPDHQLD | NH2 | Genotype C Pre-S1(2-59) |
| | (-10) | | PAFGANSNNPDWDFNPNKDHWPEANQVG | | |
| 10 | Cmyr-47 + | Myr | GLSWTVPLEWGTNLSVPNPLGFFPDHQLDP | NH2 | Genotype E or G Pre-S1(2-11) |
| | (-9) | | AFGANSNNPDWDFNPNKDHWPEANQVG | | +Genotype C Pre-S1(13-59) |
| 11 | Cplam-47 | Plam | GTNLSVPNPLGFFPDHQLDPAFGANSNNPD | NH2 | Genotype C Pre-S1(13-59) |
| | | | WDFNPNKDHWPEANQVG | | |
| 12 | Cstea-47 | Stearoyl | GTNLSVPNPLGFFPDHQLDPAFGANSNNPD | NH2 | Genotype C Pre-S1(13-59) |
| | | | WDFNPNKDHWPEANQVG | | |
| 13 | Cchol-47 | Chol | GTNLSVPNPLGFFPDHQLDPAFGANSNNPD | NH2 | Genotype C Pre-S1(13-59) |
| | | | WDFNPNKDHWPEANQVG | | |
| 14 | Amyr-47 | Myr | GTNLSVPNPLGFFPDHQLDPAFGANSNNPD | NH2 | Genotype A Pre-S1(13-59) |
| | | | WDFNPVKDDWPAANQVG | | |
| 15 | Bmyr-47 | Myr | GTNLSVPNPLGFFPDHQLDPAFKANSENPD | NH2 | Genotype B Pre-S1(13-59) |
| | | | WDLNPNKDNWPDANKVG | | |
| 16 | Dmyr-47 | Myr | GQNLSTSNPLGFFPDHQLDPAFRANTANPD | NH2 | Genotype D Pre-S1(2-48) |
| | | | WDFNPNKDTWPDANKVG | | |
| 17 | Emyr-47 | Myr | GKNISTTNPLGFFPDHQLDPAFRANTRNPD | NH2 | Genotype E Pre-S1(12-58) |
| | | | WDHNPNKDHWTEANKVG | | |
| 18 | Fmyr-47 | Myr | GQNLSVPNPLGFFPDHQLDPLFRANSSSPD | NH2 | Genotype F Pre-S1(13-59) |
| | | | WDFNTNKDSWPMANKVG | | |
| 19 | Gmyr-47 | Myr | GKNLSASNPLGFLPDHQLDPAFRANTNNPD | NH2 | Genotype G Pre-S1(12-58) |
| | | | WDFNPKKDPWPEANKVG | | |
| 20 | Hmyr-47 | Myr | GQNLSVPNPLGFFPDHQLDPLFRANSSSPD | NH2 | Genotype H Pre-S1(13-59) |
| | | | WDFNTNKDNWPMANKVG | | |

In various embodiments, the polypeptide described herein may have at least about 30%, 40%, 50%, 60%, 70%, 80%, 85%, 90%, 95%, 96%, 97%, 98%, or 99% identity to any of the polypeptides described herein. For example, the polypeptide may comprise an amino acid sequence at least about 30%, 40%, 50%, 60%, 70%, 80%, 85%, 90%, 95%, 96%, 97%, 98%, or 99% identical to any one of SEQ ID NOs: 21-40. In some embodiments, the polypeptide may comprise an amino acid sequence at least about 30%, 40%, 50%, 60%, 70%, 80%, 85%, 90%, 95%, 96%, 97%, 98%, or 99% identical to any one of SEQ ID NOs: 23 and 34-40. In some embodiments, the polypeptide may comprise an amino acid sequence at least about 30%, 40%, 50%, 60%, 70%, 80%, 85%, 90%, 95%, 96%, 97%, 98%, or 99% identical to SEQ ID NO: 23. The variants having certain sequence identity to the polypeptides described herein retain one or more biological activities of the corresponding polypeptides.

Aspects of the present disclosure also include variants of the polypeptides described herein having a native flanking amino acid sequence from the HBV L protein, such as, e.g., from the pre-S1 region of the L protein, added to the N and/or C terminus. The native flanking amino acid sequence refers to the native sequence flanking the N or C terminus of the polypeptide described herein in the pre-S1 region of the corresponding HBV genotype or any other HBV genotypes. In some embodiments, the polypeptide described herein may comprise an amino acid sequence chosen from SEQ ID NOs: 23 and 34-40, and a native flanking amino acid sequence at the N and/or C terminus derived from the pre-S1 region of any one of HBV genotypes A-H. In some embodiments, the native flanking amino acid sequence may be derived from the consensus sequence of an HBV strain with the GenBank Accession No. KC875260 (genotype A; SEQ ID NO: 41), AY220704 (genotype B; SEQ ID NO: 42), AF461363 (genotype C; SEQ ID NO: 43), AY796030 (genotype D; SEQ ID NO: 44), AB205129 (genotype E; SEQ ID NO: 45), DQ823095 (genotype F; SEQ ID NO: 46), HE981176 (genotype G; SEQ ID NO: 47), or AB179747 (genotype H; SEQ ID NO: 48). For example, the polypeptide described herein may comprise the amino acid sequence of SEQ ID NO: 23, and a native flanking amino acid sequence at the N and/or C terminus derived from the pre-S1 region of HBV genotype C. Alternatively, the polypeptide described herein may comprise the amino acid sequence of SEQ ID NO: 23, and a native flanking amino acid sequence at the N and/or C terminus derived from the pre-S1 region of any one of HBV genotypes A, B, D, E, F, G, and H. In some embodiments, the N and/or C terminus of the polypeptide described herein may independently comprise a native flanking amino acid sequence having a length of 1-10, such as, e.g., 1-8, 1-5, or 1-3 amino acids, including all integers in between these ranges. For example, the polypeptide described herein may comprise the amino acid sequence of SEQ ID NO: 23 and a native flanking amino acid sequence of 10 amino acids at the N terminus from the pre-S1 region of HBV genotype C. In other words, the polypeptide may comprise amino acids 2-59 of the pre-S1 region of HBV genotype C (SEQ ID NO: 29). As another example, the polypeptide described herein may comprise the amino acid sequence of SEQ ID NO: 23 and a native flanking amino acid sequence of 9 amino acids at the N terminus from the pre-S1 region of HBV genotype E or G. In other words, the polypeptide may comprise amino acids 13-59 of the pre-S1 region of HBV genotype C and amino acids 2-11 of the pre-S1 region of HBV genotype E or G (SEQ ID NO: 30). It will be appreciated that, any polypeptides described herein can have native flanking amino acid sequences of any length extended from the N and/or C terminus, and the resulting polypeptides retain one or more biological activities of the original polypeptides.

As used herein, “modulate” or “alter,” all used interchangeably, includes “reducing,” “decreasing,” “lowering,” “down-regulating,” or “inhibiting” one or more quantifiable parameters optionally by a defined and/or statistically significant amount. The term “modulate” also includes “enhancing,” “increasing,” “elevating,” “up-regulating,” or “promoting” one or more quantifiable parameters optionally by a defined and/or statistically significant amount.

The terms “reduce,” “decrease,” “lower,” “down-regulate,” and “inhibit,” all used interchangeably herein, mean that the level or activity of one or more chemical or biological molecules associated with metabolism is reduced below the level or activity observed in the absence of the polypeptides described herein or lower than a control polypeptide. In some embodiments, “reduce” may mean that the level or value of one or more physiological parameters that measure metabolic changes, such as, e.g., body weight, fat mass, and homeostatic model assessment (HOMA) index, are reduced below the level or activity observed in the absence of the polypeptides described herein or lower than a control polypeptide. In certain embodiments, reduction with a polypeptide described herein is below the level or activity observed in the presence of an inactive or attenuated molecule.

As used herein, homeostatic model assessment (HOMA) index may refer HOMA-IR (quantifying the level of insulin resistance) index. The value of HOMA-IR can be calculated by following the formula of: HOMA-IR=[(blood glucose expressed in mmol/L)×(serum insulin expressed in mU/L)]/22.5.

Likewise, the terms “enhance,” “increase,” “elevate,” “up-regulate,” and “promote” all used interchangeably, mean that the level or activity of one or more chemical or biological molecules associated with metabolism is increased above the level or activity observed in the absence of the polypeptides described herein or higher than a control polypeptide. In some embodiments, “enhance” may mean that the level of value of one or more physiological parameters that measure metabolic changes, such as, e.g., body weight, fat mass, and homeostatic model assessment (HOMA) index, are increased above the level or activity observed in the absence of the polypeptides described herein or higher than a control polypeptide. In certain embodiments, increase with a polypeptide described herein is above the level or activity observed in the presence of an inactive or attenuated molecule.

The terms “stabilize,” “maintain,” “sustain,” and “preserve,” are used interchangeably in connection with one or more chemical or biological molecules associated with metabolism, and mean that the level or activity of the one or more chemical or biological molecules associated with metabolism shows a minimal difference from the level or activity observed in a healthy subject or a subject who is not suffering from a metabolic disease, or from the level or activity observed in the presence of a positive control polypeptide. In some embodiments, “stabilize” may mean that the level or value of one or more physiological parameters that measure metabolic changes, such as, e.g., body weight, fat mass, and homeostatic model assessment (HOMA) index, shows a minimal difference from the level or value observed in a healthy subject or a subject who is not suffering from a metabolic disease, or from the level or value observed in the presence of a positive control polypeptide.

The polypeptides described herein can be made by chemical synthesis or by employing recombinant technology.

When recombinant procedures are selected, a synthetic gene may be constructed de novo or a natural gene may be mutated by, for example, cassette mutagenesis. The polypeptides described herein may be produced using recombinant DNA techniques. These techniques contemplate, in simplified form, taking the gene, either natural or synthetic, encoding the peptide; inserting it into an appropriate vector; inserting the vector into an appropriate host cell; culturing the host cell to cause expression of the gene; and recovering or isolating the peptide produced thereby. In some embodiments, the recovered peptide is then purified to a suitable degree.

For example, the DNA sequence encoding a polypeptide described herein is cloned and manipulated so that it may be expressed in a convenient host. DNA encoding parent polypeptides can be obtained from an HBV genomic library, from cDNA derived from mRNA from cells expressing the polypeptide, or by synthetically constructing the DNA sequence. The parent DNA is then inserted into an appropriate plasmid or vector which is used to transform a host cell. In general, plasmid vectors containing replication and control sequences which are derived from species compatible with the host cell are used in connection with those hosts. The vector ordinarily carries a replication site, as well as sequences which encode proteins or peptides that are capable of providing phenotypic selection in transformed cells. The vector may be those commonly used in the art, or constructed using standard techniques by combining functional fragments of the vectors commonly used in the art.

The host cell may be prokaryotic or eukaryotic. For example, prokaryotic host cells may include E. coli, Bacillus subtilis, and other enterobacteriaceae such as, e.g., Salmonella typhimurium or Serratia marcesans, and various Pseudomonas species. In addition to prokaryotes, eukaryotic organisms, such as yeast cultures, or cells derived from multicellular organisms, such as insect or mammalian cell cultures, may be used. Examples of such eukaryotic host cell lines include VERO and HeLa cells, Chinese Hamster Ovary (CHO) cell lines, W138, 293, BHK, COS-7, and MDCK cell lines.

In some embodiments, the polypeptides described herein may be prepared using solid-phase synthesis, or other equivalent chemical syntheses known in the art. In some embodiments, solid-phase synthesis is initiated from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a BHA resin or MBHA resin. The amino acids are coupled to the peptide chain using techniques well known in the art for the formation of peptide bonds. One method involves converting the amino acid to a derivative that will render the carboxyl group more susceptible to reaction with the free N-terminal amino group of the peptide fragment. For example, the amino acid can be converted to a mixed anhydride by reaction of a protected amino acid with ethylchloroformate, phenyl chloroformate, sec-butyl chloroformate, isobutyl chloroformate, pivaloyl chloride or like acid chlorides. Alternatively, the amino acid can be converted to an active ester such as a 2,4,5-trichlorophenyl ester, a pentachlorophenyl ester, a pentafluorophenyl ester, a p-nitrophenyl ester, a N-hydroxysuccinimide ester, or an ester formed from 1-hydroxybenzotriazole. Another coupling method involves use of a suitable coupling agent such as N,N′-dicyclohexylcarbodiimide or N,N′-diisopropylcarbodiimide.

In some embodiments, the α-amino group of each amino acid employed in the peptide synthesis may be protected during the coupling reaction to prevent side reactions involving their active α-amino function. For example, certain amino acids that contain reactive side-chain functional groups (e.g., sulfhydryl, amino, carboxyl, and hydroxyl) may be protected with suitable protecting groups to prevent a chemical reaction from occurring at that site during both the initial and subsequent coupling steps. The selection of a suitable side-chain protecting group is within the skill of the art. The protecting group will be readily removable upon completion of the desired amino acid peptide under reaction conditions that will not alter the structure of the peptide chain.

After removal of the α-amino protecting group, the remaining α-amino and side-chain protected amino acids are coupled stepwise within the desired order. As an alternative to adding each amino acid separately in the synthesis, some may be coupled to one another prior to addition to the solid-phase synthesizer. The selection of an appropriate coupling reagent is within the skill of the art.

Each protected amino acid or amino acid sequence is introduced into the solid-phase reactor in excess, and the coupling is suitably carried out in a medium of dimethylformamide (DMF) or CH2Cl2 or mixtures thereof. If incomplete coupling occurs, the coupling procedure is repeated before removal of the N-amino protecting group prior to the coupling of the next amino acid. The success of the coupling reaction at each stage of the synthesis may be monitored. The coupling reactions can be performed automatically using well known methods, for example, a BIOSEARCH 9500™ peptide synthesizer.

Upon completion of the desired peptide sequence, the protected peptide must be cleaved from the resin support, and all protecting groups must be removed. The cleavage reaction and removal of the protecting groups is suitably accomplished simultaneously or stepwise. When the resin support is a chloro-methylated polystyrene resin, the bond anchoring the peptide to the resin is an ester linkage formed between the free carboxyl group of the C-terminal residue and one of the many chloromethyl groups present on the resin matrix. It will be appreciated that the anchoring bond can be cleaved by reagents that are known to be capable of breaking an ester linkage and of penetrating the resin matrix. It will also be recognized that the polypeptides may be modified (such as, e.g., modified at the N-terminus with a hydrophobic group, including, e.g., myristic acid, palmitic acid, stearic acid, oleic acid, linoleic acid, cholesterol, arachidonic acid; modified at the C-terminus by amidation (amination), isopentanediolization, or other stabilizing C-terminal modification) either before or after the polypeptide is cleaved from the support.

Purification of the polypeptides of the invention may be achieved using conventional procedures such as preparative HPLC (including reversed phase HPLC) or other known chromatographic techniques such as gel permeation, ion exchange, partition chromatography, affinity chromatography (including monoclonal antibody columns) or countercurrent distribution.

II. Pharmaceutical Compositions

The present disclosure also provides compositions, including pharmaceutical compositions, comprising a polypeptide described herein. In certain embodiments, the composition may comprise any one or more polypeptides described herein. In some embodiments, the composition may further comprise a suitable pharmaceutically acceptable carrier.

A “pharmaceutically acceptable carrier” refers to an inactive ingredient, such as, e.g., solid, semisolid, or liquid filler, diluent, encapsulating material, formulation auxiliary, excipient, or carrier, for use with a therapeutic agent that together comprise a “pharmaceutical composition” for administration to a subject. A pharmaceutically acceptable carrier is non-toxic to recipients at the dosages and concentrations employed, and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

Pharmaceutical compositions of the polypeptides described herein may be prepared by mixing such polypeptide having the desired degree of purity with one or more optional pharmaceutically acceptable carriers. Pharmaceutically acceptable carriers may include, e.g.: buffers (such as, e.g., phosphate, citrate, and other organic acids); antioxidants (such as, e.g., ascorbic acid and methionine); preservatives (such as, e.g., octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (such as, e.g., less than about 10 residues) polypeptides; proteins (such as, e.g., serum albumin, gelatin, or immunoglobulins); hydrophilic polymers (such as, e.g., polyvinylpyrrolidone); amino acids (such as, e.g., glycine, glutamine, asparagine, histidine, arginine, or lysine); monosaccharides, disaccharides, and other carbohydrates including glucose, mannose, or dextrins; chelating agents (such as, e.g., EDTA); sugars (such as, e.g., sucrose, mannitol, trehalose or sorbitol); salt-forming counter-ions (such as, e.g., sodium); metal complexes (such as, e.g., Zn-protein complexes); and/or non-ionic surfactants (such as, e.g., polyethylene glycol (PEG)).

Exemplary pharmaceutical carriers may also include binding agents (such as, e.g., pregelatinized maize starch, polyvinylpyrrolidone or hydroxypropyl methylcellulose, etc.); fillers (such as, e.g., lactose and other sugars, microcrystalline cellulose, pectin, gelatin, calcium sulfate, ethyl cellulose, polyacrylates or calcium hydrogen phosphate, etc.); lubricants (such as, e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (such as, e.g., starch, sodium starch glycolate, etc.); and wetting agents (such as, e.g., sodium lauryl sulphate, etc.).

Exemplary pharmaceutically acceptable carriers may further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). In some embodiments, a sHASEGP may be combined in the pharmaceutical composition with one or more additional glycosammoglycanases, such as, e.g., chondroitinases.

The pharmaceutical compositions may also comprise more than one active ingredient suitable for the particular indication being treated, for example, those with complementary activities that do not adversely affect each other. Such active ingredients may be suitably present in combination in amounts that are effective for the purpose intended.

In some embodiments, the active ingredients may be entrapped in microcapsules prepared, for example, by coacervation techniques or by interfacial polymerization, such as, e.g., hydroxymethylcellulose or gelatin-microcapsules and poly-(methylmethacylate) microcapsules, respectively, in colloidal drug delivery systems (such as e.g., liposomes, albumin microspheres, microemulsions, nano-particles and nanocapsules), or in macroemulsions.

In some embodiments, the pharmaceutical composition may comprise sustained-release preparations. Suitable examples of sustained-release preparations include, e.g., semipermeable matrices of solid hydrophobic polymers containing the polypeptides described herein, which matrices may be in the form of shaped articles, such as, e.g., films or microcapsules.

In some embodiments, the pharmaceutical compositions may be used for in vivo administration and may be sterile. Sterility may be readily accomplished, e.g., by filtration through sterile filtration membranes.

The pharmaceutical compositions may be formulated into any of many possible dosage forms, such as, e.g., tablets, capsules, gel capsules, powders, or granules. The pharmaceutical compositions may also be formulated as solutions, suspensions, emulsions, or mixed media. In some embodiments, the pharmaceutical compositions may be formulated as lyophilized formulations or aqueous solutions.

In some embodiments, the pharmaceutical compositions may be formulated as a solution. For example, the polypeptides described herein may be administered in an unbuffered solution, such as, e.g., in saline or in water. In some embodiments, the polypeptides may also be administered in a suitable buffer solution. For example, the buffer solution may comprise acetate, citrate, prolamine, carbonate, or phosphate, or any combination thereof. In some embodiments, the buffer solution may be phosphate buffered saline (PBS). The pH and osmolality of the buffer solution containing the polypeptides can be adjusted to be suitable for administering to a subject.

In some embodiments, the pharmaceutical compositions may be formulated as suspensions in aqueous, non-aqueous, or mixed media. Aqueous suspensions may further contain substances which increase the viscosity of the suspension including, for example, sodium carboxymethylcellulose, sorbitol and/or dextran. The suspension may also contain stabilizers.

In some embodiments, the pharmaceutical compositions may be formulated as emulsions. Exemplary emulsions include heterogeneous systems of one liquid dispersed in another in the form of droplets usually exceeding 0.1 μm in diameter. Emulsions may contain additional components in addition to the dispersed phases, and the active drug which may be present in a solution in the aqueous phase, the oily phase, or itself as a separate phase. Microemulsions are also included as an embodiment of the present disclosure. In some embodiments, the pharmaceutical compositions may also be formulated as liposomal formulations.

III. Methods of Use

Embodiments of the present disclosure include therapeutic and pharmaceutical uses of the polypeptides described herein. Therefore, in one aspect, use of the polypeptides described herein as a medicament or in preparing medicaments is provided. In another aspect, use of the polypeptides described herein in treating nonalcoholic fatty liver disease is provided. A method of treating nonalcoholic fatty liver disease in a subject is also provided, comprising administering to the subject a therapeutically effective amount of the polypeptides described herein or of a pharmaceutical composition comprising such polypeptide. In certain embodiments, methods and uses described herein may further comprise administering to the subject a therapeutically effective amount of at least one additional therapeutic agent.

Nonalcoholic fatty liver disease (NAFLD) described herein includes but is not limited to simple fatty liver (SFL), nonalcoholic steatohepatitis (NASH), fatty liver fibrosis and cirrhosis, especially includes NASH.

“Patient” and “subject” may be used interchangeably to refer to an animal, such as a mammal or a human, being treated or assessed for a disease, disorder, or condition, at risk of developing a disease, disorder, or condition, or having or suffering from a disease, disorder, or condition. In some embodiments, such disease, disorder, or condition may include nonalcoholic fatty liver disease (NAFLD) described herein.

The term a “therapeutically effective amount” or “effective amount” of a polypeptide described herein or a composition comprising such polypeptide refers to an amount effective in the prevention or treatment of a disorder for the treatment of which the polypeptide or composition is effective. The “therapeutically effective amount” or “effective amount” may vary depending on the polypeptide, the route of administration, the disease and its severity, and the health, age, weight, family history, genetic makeup, stage of pathological processes, the types of preceding or concomitant treatments, if any, and other individual characteristics of the subject to be treated.

In various embodiments, the term “treatment” includes treatment of a subject (e.g. a mammal, such as a human) or a cell to alter the current course of the subject or cell. Treatment includes, e.g., administration of a polypeptide described herein or a pharmaceutical composition comprising such polypeptide, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition or the associated symptoms. In some embodiments, the term “treatment” may include improving at least one symptom or measurable parameter of nonalcoholic fatty liver disease. It will be apparent to one of skill in the art which biological and/or physiological parameters can be used to access the pathological process of metabolic disease. Such pathological processes or symptoms may include, e.g., excessive or increased levels compared with healthy subjects of one or more chemical or biological molecules associated with metabolism, such as, e.g., glucose, triglyceride, cholesterol, free fatty acids, bile acids, amino acids, hormones, including, e.g., insulin, LDL-C, HDL-C, HbA1c, blood urea nitrogen, and minerals; or of one or more physiological parameters that measure metabolic changes, such as, e.g., glycemia, blood pressure, body weight, fat mass, body mass index (BMI), inflammation, atherosclerosis index (AI), heart index, kidney index, total fat index, and homeostatic model assessment (HOMA) index.

The terms “administering,” or “administer” include delivery of the polypeptide described herein to a subject either by local or systemic administration. Administration may be topical (including ophthalmic and to mucous membranes including vaginal and rectal delivery), pulmonary (e.g., by inhalation or insufflation of powders or aerosols, including by nebulizer, intratracheal, intranasal), epidermal, transdermal, oral, or parenteral. Parenteral administration includes intravenous, subcutaneous, intraperitoneal, or intramuscular injection or infusion; or intracranial, e.g., intrathecal or intraventricular, administration.

In a further aspect, the present disclosure provides for the use of the polypeptides described herein in the manufacture or preparation of a medicament. In some embodiments, the medicament may be useful for treatment of the nonalcoholic fatty liver disease described herein.

In certain embodiments, the methods and uses described herein may further comprise administering to the subject an effective amount of at least one additional therapeutic agent. In certain embodiments, the additional therapeutic agent may be for preventing and/or treating one or more diseases associated with the nonalcoholic fatty liver disease described herein, such as, e.g., one or more diseases associated with diabetes or hyperlipidemia. In certain embodiments, the additional therapeutic agent may be for preventing and/or treating cardiovascular diseases (e.g., atherosclerotic diseases). In certain embodiment, the additional therapeutic agent may be for reducing the risk of recurrent cardiovascular events. In certain embodiments, the additional therapeutic agent may be for preventing and/or treating heart diseases, kidney impairment, or obesity. The polypeptides described herein can be used either alone or in combination with other agents in a therapy. For instance, any of the polypeptides described herein may be administered before, concurrently with, or after administration of at least one additional therapeutic agent. In certain embodiments, the additional therapeutic agent may be chosen from e.g., an antihyperlipidemic agent, an antihyperglycemic agent, an antidiabetic agent, an antiobesity agent, and a bile acid analogue.

In some embodiment, the antihyperglycemic agent may be chosen from, e.g., a biduanide (e.g., metformin, phenformin, and buformin), insulin (e.g., regular human insulin, NPH insulin, insulin aspart, insulin lispro, insulin glargine, insulin detemir, and insulin levemir), a glucagon-like peptide 1 receptor agonist (GLP-1RA; e.g., albiglutide, dulaglutide, exenatide, liraglutide, lixisenatide, and extended-release glucagon), a sodium-glucose cotransporter 2 inhibitor (SGLR2I; e.g., canagliflozin, empagliflozin, dapagliflozin, empagliflozin, and ipragliflozin), a dipeptidyl peptidase 4 inhibitor (DPP4I; e.g., bromocriptine, sitagliptin, vildagliptin, saxagliptin, linagliptin, anagliptin, teneligliptin, alogliptin, trelagliptin, gemigliptin, dutogliptin, omarigliptin, berberine, and lupeol), an α-glucosidase inhibitor (AGI; e.g., miglitol, acarbose, and voglibose), a thiazolidinedione (TZD; e.g., pioglitazone, rosiglitazone, lobeglitazone, troglitazone, ciglitazone, darglitazone, englitazone, netoglitazone, rivoglitazone, and mifepristone), a meglitinide (e.g., repaglinide, nateglinide, and mitiglinide), a sulfonylurea (SU; e.g., carbutamide, acetohexamide, chlorpropamide, tolbutamide, tolazamide, glipizide (glucotrol), gliclazide, glibenclamide, glyburide (e.g. Micronase), glibornuride, gliquidone, glisoxepide, glyclopyramide, glimepiride, amaryl, and glimiprime), an amylin analogue (e.g., pramlinitide), a proprotein convertase subtilisin/kexin type 9 inhibitor (PCSK9I; e.g., evolocumab, bococizumab, alirocumab, 1D05-IgG2, RG-7652, LY3015014, RNAi therapeutic ALN-PCS02, AMG-145, and REGN727/SAR236553), a glucokinase activator (GKA; e.g., MK-0941, RO-28-1675, and AZD1656), a PPAR agonist/modulator, a glucagon receptor antagonist, a C—C chemokine receptor type 2 (CCR2) antagonist, an Interleukin-1 modulator, a G-protein coupled receptor agonist, a gastrointestinal peptide agonist other than GLP-1, an SGLT1 and dual SGLT1/SGLT2 inhibitor (excluding an SGLT2-only inhibitor), an llbeta-HSD1 inhibitor, a diacylglycerol acyltransferase (DGAT)-1 inhibitor, a cannabinoid, a hepatic carnitine palmitoyltransferase 1 (CPT1) inhibitor, a fibroblast growth factor (FGF)-21 agonist, a glucocorticoid receptor antagonist, a heat shock protein (HSP) inducer, a melanocortin-4 receptor (MC4R) agonist, a tetrahydrotriazin containing oral antidiabetic, glimin, a protein tyrosine phosphatase 1B (PTP1B) inhibitor, a sirtuinl (SIRT1) activator, and a microbiome modulator.

In some embodiment, the additional therapeutic agent is an antihyperlipidemic agent and may be chosen from, e.g., a statin (e.g., HMG-CoA reductase inhibitor; e.g., smvastatin, atorvastatin, rosuvastatin, pravastatin, pitavastatin, lovastatin, atorvastatin, fluvastatin, cerivastatin, mevastatin, pantethine, elastase, and probucol), a fibric acid (e.g., bezafibrate (e.g., Bezalip), ciprofibrate (e.g., Modalim), clofibrate, gemfibrozil (e.g., Lopid), fenofibrate (e.g., TriCor), clinofibrate (e.g., Lipoclin), lifibrate, alufibrate, simfibrate, etofylline clofibrate, and gemfibrozil), a nicotinic acid (e.g., niacin, inositol hexanicotinate, nicotinamide, and acipimox), a bile acid sequestrant (e.g., cholestyramine (e.g., Questran®), colesevelam (e.g., Welchol®), colestipol (e.g., Colestid®), polidexide, dholestyramine, and divistyramine), ezetimibe (e.g., Zetia), a proprotein convertase subtilisin/kexin type 9 inhibitor (PCSK9I; e.g., evolocumab, bococizumab, alirocumab, 1D05-IgG2, RG-7652, LY3015014, RNAi therapeutic ALN-PCS02, AMG-145, and REGN727/SAR236553), a microsomal triglyceride transfer protein inhibitor (MTTPI; e.g., lomitapide and JTT-130), an apolipoprotein B inhibitor (apoBl; e.g., mipomersen (e.g., Kynamro)), a diacylglycerol acyltransferase 1 (DGAT1) inhibitor (e.g., pradigastat), an angiopoietin-like protein 3 inhibitor (e.g., REGN1500), a cholesteryl ester transfer protein (CETP) inhibitor (e.g., anacetrapib and evacetrapib), a peroxisome proliferator-activated receptor (PPAR) a/y agonist, an acyl-CoA inhibitor, an incretin mimetics inhibitor, an angiopoietin-like protein 3 (ANGPTL3) inhibitor, an angiopoietin-like protein 4 (ANGPTL4) inhibitor, an apoC-III-targeted inhibitor, and a selective peroxisome proliferator-activated receptor modulator (SPPARM).

In some embodiment, the additional therapeutic agent is an antiobesity agent and may be chosen from, e.g., orlistat (e.g., Xenical), lorcaserin (e.g., Belviq), phentermine, topiramate, diethylpropion, phendimetrazine, benzphetamine, and a combination of phendimetrazine and benzphetamine.

In some embodiment, the additional therapeutic agent is a bile acid analogue and may be chosen from, e.g., obeticholic acid, ursodeoxycholic acid, and cholylsarcosine.

In some embodiments, the additional therapeutic agent may also be chosen from, e.g., a farnesoid X receptor (FXR) agonist, an FXR inhibitor, a transemembrane G protein-coupled receptor 5 (TGR5) agonist, and a TGR5 inhibitor.

In some embodiments, the additional therapeutic agent may be chosen from insulin, metformin, sitagliptin, colesevelam, glipizide, simvastatin, atorvastatin, ezetimibe, fenofibrate, nicotinic acid, orlistat, lorcaserin, phentermine, topiramate, obeticholic acid, and ursodeoxycholic acid.

Such combination therapies described herein may encompass combined administration (where two or more therapeutic agents are included in the same or separate formulations), and separate administration, in which case, administration of the polypeptides described herein can occur prior to, simultaneously, and/or following, administration of the additional therapeutic agent.

The polypeptides described herein (and any additional therapeutic agent) can be administered by any suitable means, including parenteral, intrapulmonary, and intranasal, and, if desired for local treatment or intralesional administration. In some embodiments, the polypeptides described herein may be parenterally administered. Parenteral administration may include intramuscular, intravenous, intraarterial, intraperitoneal, or subcutaneous administration. In some embodiments, the polypeptides described herein may be administered subcutaneously. In some embodiments, the polypeptides described herein may be administered intravenously. Dosing can be by any suitable route, such as, e.g., by injections or infusions, such as intravenous or subcutaneous injections or infusions, depending in part on whether the administration is brief or chronic. Various dosing schedules including e.g. single or multiple administrations over various time-points, bolus administration, and pulse infusion are also contemplated.

The polypeptides described herein would be formulated, dosed, and administered in a fashion consistent with common medical practice. Factors for consideration in this context may include, e.g., the particular disorder being treated, the particular mammal being treated, the clinical condition of the individual patient, the cause of the disorder, the site of delivery of the agent, the method of administration, the scheduling of administration, and other factors known to medical practitioners. The polypeptides described herein need not be but can be optionally formulated with one or more agents currently used to prevent or treat the disorder in question. The effective amount of such other agents depends on the amount of the polypeptide described herein present in the formulation, the type of disorder or treatment, and other factors discussed above.

For the prevention or treatment of disease, the appropriate dosage of a polypeptide described herein (when used alone or in combination with one or more other additional therapeutic agents) may depend on the type of disease to be treated, the severity and course of the disease, whether the polypeptide is administered for preventive or therapeutic purposes, previous therapy, the patient's clinical history and response to the polypeptide, and the discretion of the attending physician. The polypeptides described herein may be suitably administered to the patient at one time or over a series of treatments. Depending on the type and severity of the disease, the polypeptide described herein may be administered to the patient, for example, by one or more separate administrations, or by continuous infusion. For repeated administrations over several days or longer, depending on the condition, the treatment may be sustained until a desired suppression of disease symptoms occurs. The polypeptide described herein may be administered intermittently, e.g. every day, every two days, every three days, every week, or every two or three weeks (e.g. such that the patient receives from more than one, such as, e.g., about two to about twenty, or e.g. about six doses of the polypeptide). An initial higher loading dose, followed by one or more lower doses, may be administered.

In certain embodiments, a flat-fixed dosing regimen may be used to administer the polypeptide described herein to a subject. However, other dosage regimens may also be useful depending on the factors discussed above. The progress of this therapy can be easily monitored by conventional techniques and assays for the disease or condition treated.

The following Examples may be used for illustrative purposes and should not be deemed to narrow the scope of the invention.

### EXAMPLE

In this example, a mouse nonalcoholic steatohepatitis (NASH) model was used to evaluate the effect of hepalatide (L47) on pathological changes and metabolism level of NASH, and to determine whether L47 can improve the pathological changes of NASH, so as to provide reference for pharmacodynamic evaluation of L47 before clinical treatment.

I. Experimental Materials and Methods

1. Experimental Materials Hepalatide (L47); preparation: designated L47 was placed into a 15 ml centrifuge tube, and 1×PBS buffer of a designated volume was added, the mixture is shaken well and is used immediately.

Preparation of PBS Buffer Stock:

liquid A: 0.2M Na₂HPO₄: 71.6 g of Na₂HPO₄-12H₂O and 8 g of NaCl were dissolved in 1000 ml water;

liquid B: 0.2M NaH₂PO₄: 31.2 g of NaH₂PO₄-2H₂O and 8 g of NaCl were dissolved in 1000 ml water;

20×PBS buffer: 81 ml of liquid A and 19 ml of liquid B were mixed to form a 100 ml mixture;

1×PBS buffer: 50 ml of 20×PBS buffer was diluted to 1 L.

2. Experimental Animals

Strain: C57BL6 mouse; grade: SPF grade; week age: 6-8 weeks; gender: male; source: Nanjing Biomedical Research Institute of Nanjing University, SOCK (Jiangsu) 2015-0001, animal quality certificate No.: 201604593, 201605069 respectively.

3. Experimental Conditions

License of Department of Experimental Animal Science of Fudan University: SOCK (Shanghai) 2014-0004.

Drinking water: tap water was filtered and sterilized, then placed in a high-pressure sterilized water bottle and freely accessed. Drinking water was replaced twice per week.

High sugar water: 18.9 g of glucose and 23.1 g of fructose were dissolved in 1 L of drinking water.

General feed (basic feed with 12% fat): complete nutritious mouse pellet feed sterilized by Co⁶⁰ irradiation (Department of Experimental Animal Science of Fudan University).

High fat feed: purchased from American Research Diet Company, Cat No.: D12492, fat: 60%. Feeding mode: four or five C57BL6 mice per cage were fed in IVC with free diet, enough water and feed, and beddings were replaced every two days. Mice were weighed every week and the feed consumption of each cage of mice was recorded. During the whole experiment, experimental feeding and operation for mice were approved by the ethics committee of School of Basic Medical Sciences, Fudan University.

4. Animal Grouping and Treatment

4.1 Dosage and Group

4.1.1 Normal control group (Control): common block feed+common water, 14 mice in total, wherein 6 mice were sacrificed at week 8, and 8 mice were sacrificed at week 16.

4.1.2 Model group (HFC-F/G): high fat feed+high sugar water, 9 mice in total, wherein 3 mice were sacrificed at week 8, and 6 mice were sacrificed at week 16.

4.1.3 Low dose group 1 (HFC-F/G+HL1): high fat feed+high sugar water+L47 20 mg/kg (10 mg/kg*2/d, i.e. once in the morning and once in the afternoon), 10 mice in total, wherein 3 mice were sacrificed at week 8, and 7 mice were sacrificed at week 16.

4.1.4 High dose group 1 (HFC-F/G+HH1): high fat feed+high sugar water+L47 60 mg/kg (30 mg/kg*2/d, i.e. once in the morning and once in the afternoon), 10 mice in total, wherein 3 mice were sacrificed at week 8, and 7 mice were sacrificed at week 16.

4.1.5 Low dose group 2 (HFC-F/G+HL2): high fat feed+high sugar water+L47 20 mg/kg (10 mg/kg*2/d, i.e. once in the morning and once in the afternoon, with pharmacologic intervention started 8 weeks after initiation of the experiment), 5 mice in total, which are sacrificed after 16 weeks.

4.1.6 High dose group 2 (HFC-F/G+HH2): high fat feed+high sugar water+L47 60 mg/kg (30 mg/kg*2/d, i.e. once in the morning and once in the afternoon, with pharmacologic intervention started 8 weeks after initiation of the experiment), 5 mice in total, which are sacrificed after 16 weeks.

The concentration of L47 in low dose group was 5 mg/ml; the concentration of L47 in high dose group was 10 mg/ml.

Route of administration: subcutaneous injection.

5. Reagents and Instruments

5.1 Main Reagents

5.1.1 Chloral hydrate, provided by Sinopharm Chemical Reagent Co., Ltd;

5.1.2 Na₂HPO₄-12H₂O, Lot No.: 20150603, Sinopharm Chemical Reagent Co., Ltd;

5.1.3 NaH₂PO₄-2H₂O, Lot No.: 20120330, Sinopharm Chemical Reagent Co., Ltd;

5.1.4 Glucose, Cat No.: G8270-5KG, Sigma Company;

5.1.5 Fructose, Cat No.: F3510-5KG, Sigma Company;

5.1.6 10% formalin, Cat No.: HT501128-4L, Sigma Company;

5.1.7 OTC, Cat No.: 4583, Sakura Company;

5.1.8 75% ethanol, Lot No.: 20141216, Sinopharm Chemical Reagent Co., Ltd;

5.1.9 Blood glucose test paper, Lot No.: 1616530210, Omron.

5.2 Test Kits

5.2.1 Serum Biochemical Kit:

Total cholesterol (TC), triglyceride (TG), total bilirubin (TBIL), high-density lipoprotein cholesterol (HDL-C), low-density lipoprotein cholesterol (LDL-C), alkaline phosphatase (ALP), albumin (ALB), aspartate aminotransferase (AST) and alanine aminotransferase (ALT) were provided by Shanghai Adicon Company and tested by automatic biochemical analyzer.

Total bile acid (TBA), very low density lipoprotein (VLDL): provided by Nanjing Jiancheng Bioengineering Institute.

5.2.2 Liver Biochemical Kit:

Hydroxyproline and free fatty acid (FFA) kits (Cat No.: A030-3 and A042-1, respectively) were provided by Nanjing Jiancheng Bioengineering Research Institute;

Triglyceride (TG) and cholesterol (TC) (Cat No.: E1013-105 and E1015 respectively) were provided by Applygen Technologies Inc.

5.2.3 Insulin ELISA Test Kit, Cat No.: EZRMI-13K, Millipore Company.

5.3 Main Instruments

5.3.1 Electronic analytical balance, Sartorius Company;

5.3.2 BAT-1B/131126AA-18 liquid nitrogen irrigation, MVE Company;

5.3.3 H2O-I-1-T ultra-purified water purification unit, Sartorius Company;

5.3.4 Flex station 3 multifunction board reader-calcium flow detection workstation;

5.3.5 TS100 inverted microscope, Nikon Eclipse Company;

5.3.6 VB-95 autoclave, Systec;

5.3.7 HGM-112 blood glucose meter, Omron.

6. Observation Indicators

6.1 General Observation

Each experimental group was provided with sufficient feed and water daily with free diet, and water was changed twice a week for sugar water-added group, and the remaining amount of feed was weighed and added to 150 g each week for each group in addition to the normal control group, and body weight of mice was weighed each week for each group.

6.2 Gross Anatomical Observation

At week 8 and week 16, liver was separated by dissection after anesthesia and blood collection, and pathological changes of liver tissues and accumulation of abdominal fat were grossly observed by the naked eye.

6.3 Observation and Determination of Body Fat Deposition

Quantitative CT was used to locate and quantify subcutaneous, visceral and brown fat in mice at week 16.

6.4 Detection of Blood Glucose, Glucose Tolerance and Insulin Resistance Index

At week 8 and week 16, fasting blood glucose and glucose tolerance (subcutaneous injection of glucose: 2.5 mg/g, preparation concentration: 250 mg/ml) were measured in mice, blood glucose levels were measured at 0, 30, 60, 90 and 120 min after the injection of glucose, and insulin resistance index was calculated according to the fasting insulin level.

6.5 Blood Biochemistry Test

At week 8 and week 16, blood was taken from fasted mice (fasted for 12 hours) after anesthesia, and levels of serum TG, TC, ALB, ALP, ALT, AST, HDL-C, LDL-C, VLDL, TBIL, TBA, insulin (Ins) were measured.

6.6 Liver Biochemistry Test

At week 8 and week 16, liver was taken and levels of TG, TC, free fatty acid (FFA), hydroxyproline (HYP) were measured.

6.7 Histological Observation

At week 8 and week 16, hematoxylin eosin (HE) staining, trichrome staining (Masson staining), Oil Red 0 staining and apoptosis (TUNEL) staining were performed on the liver tissue sections of mice.

7. Data Processing

SPSS software was used for statistical analysis, all data were represented by mean±standard error (mean±SEM), ANOVA variance was used to evaluate the experimental results of measurement data, and LSD test was used for comparison of two groups, and the statistical results were correct to two decimal places.

II. Experimental Results

1. Effect of L47 on Liver Weight of C57BL6 Mice

It can be seen from FIG. 1 that at week 8, there was no significant difference in the liver weight of each group and there was an increasing trend in the model group.

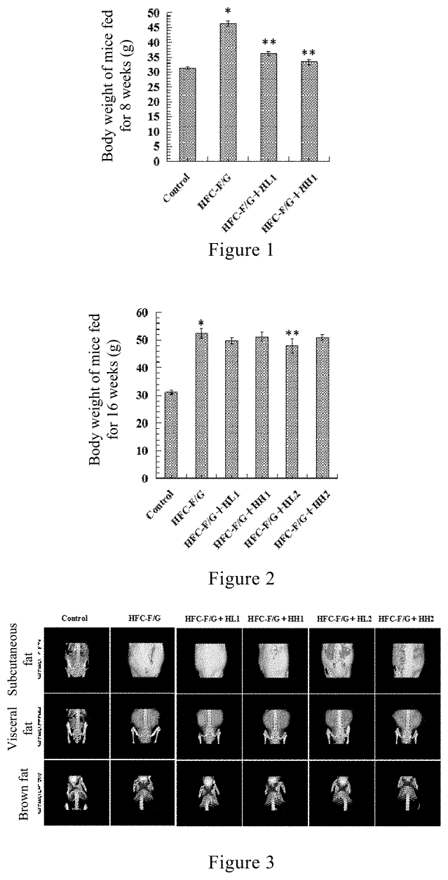

It can be seen from FIG. 2 that at week 16, the liver weights of the model group increased significantly as compared with the normal control group (p<0.001), and the liver weights of the low dose group 2 (HL2) decreased significantly as compared with the model group (p<0.05).

2. Fat Distribution and Quantification of Each Group at Week 16

After micro CT scanning, the data analysis results are shown in FIG. 3. At week 16, subcutaneous fat, visceral fat and brown fat of the model group were all significantly more than those of the normal control group. Subcutaneous fat of the high dose group 1 (HH1), the low dose group 2 (HL2) and the high dose group 2 (HH2) were decreased as compared with the model group. There was no significant difference in visceral fat and brown fat among each group except for the normal control group.

3. Histological Changes in Liver at Week 8 and Week 16

3.1 HE Staining at Week 8 and Week 16

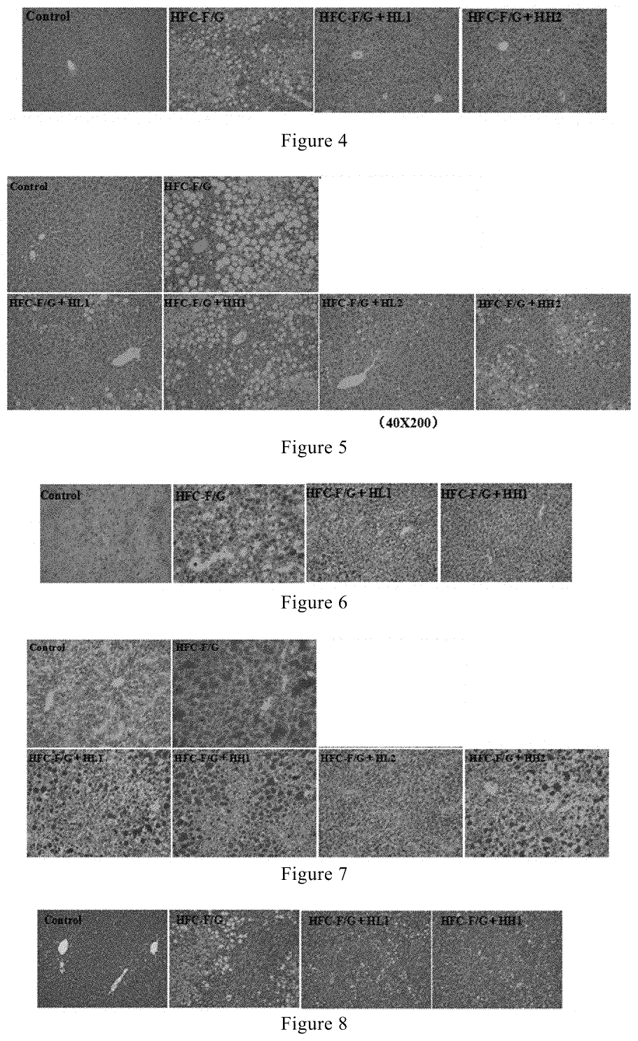

It can be seen from FIG. 4 that at week 8, compared with the mouse livers of the normal control group, fat vacuoles of different sizes were formed in hepatocytes of the model group with increased inflammatory cells; while in the low and high dose groups, only less and smaller fat vacuoles were formed, with less inflammatory cells.

It can be seen from FIG. 5 that at week 16, compared with the mouse livers of the normal control group, severe steatosis was observed in the model group and the high dose group 1 (HH1), and the inflammatory cell infiltration was obvious, while other groups showed slight steatosis and inflammatory response.

3.2 Oil Red O Staining at Week 8 and Week 16

It can be seen from FIG. 6 that at week 8, compared with the mouse livers of the normal control group, larger fat droplet vacuoles were observed in the model group, while only smaller and less vacuoles in the treatment groups.

It can be seen from FIG. 7 that at week 16, compared with the mouse livers of the normal control group, large fat vacuoles were formed in the hepatocytes of the model group, which were larger and more than that at week 8. Compared with the model group, fat accumulations of the low dose group 1 (HL1) and the high dose group 1 (HH1) were only slightly reduced, but the fat accumulations of the low dose group 2 (HL2) and the high dose group 2 (HH2) were significantly reduced, forming fewer and smaller fat vacuoles.

3.3 Masson Staining at Week 8 and Week 16

It can be seen from FIG. 8 that at week 8, compared with the mouse livers of the normal control group, more fiber cords were observed in the model group, while no significant difference were observed between other groups and the model group.

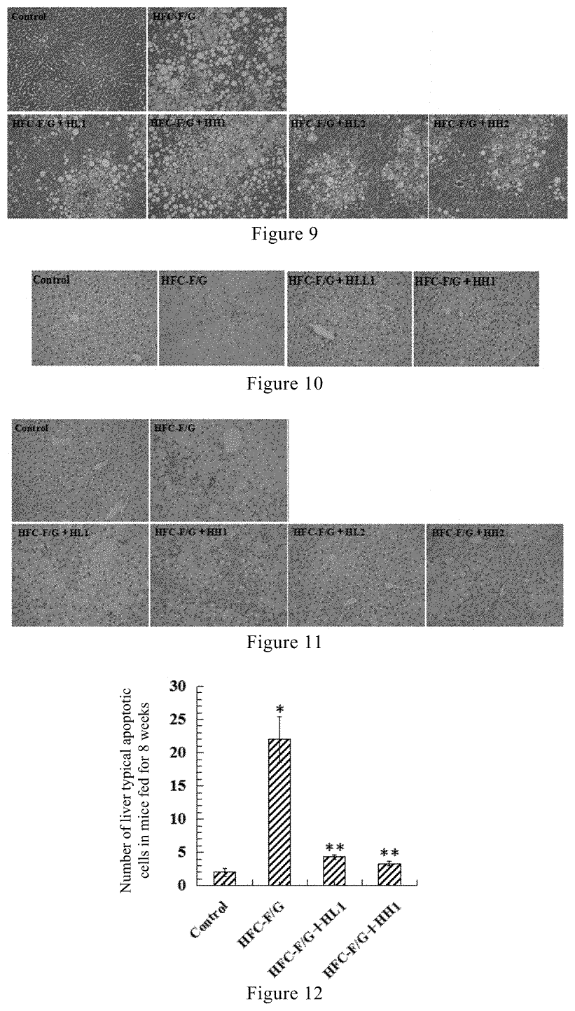

It can be seen from FIG. 9 that at week 16, compared with the mouse livers of the normal control group, more and more obvious fibrous cords were observed in the model group, there was no significant difference between the high dose group 1 (HH1) and the model group, and between the low dose group 1 (HL1) and the model group, while the fibrous depositions in the high dose group 2 (HH2) and the low dose group 2 (HL2) were decreased as compared with the model group.

3.4 TUNEL Staining and Quantification at Week 8 and Week 16

3.4.1 TUNEL Staining Histology

It can be seen from FIG. 10 that at week 8, compared with the normal control group, a large number of typical apoptotic cells were formed in the mouse livers of the model group, and the apoptosis in the treatment groups were effectively improved.

It can be seen from FIG. 11 that at week 16, compared with the normal control group, a large number of typical apoptotic cells were formed in the mouse livers of the model group, and the apoptosis in the low dose group 2 (HL2) and the high dose group 2 (HH2) were effectively improved. However, no improvement in apoptosis was observed in the low dose group 1 (HL1) and the high dose group 1 (HH1).

3.4.2 TUNEL Semi-Quantitative Counting

It can be seen from FIG. 12 that at week 8, compared with the normal control group, the number of typical TUNEL staining positive cells in the mouse livers of the model group increased significantly (p<0.001, p<0.01). Compared with the model group, the number of apoptotic bodies in the treatment groups was significantly decreased (p<0.001, p<0.001).

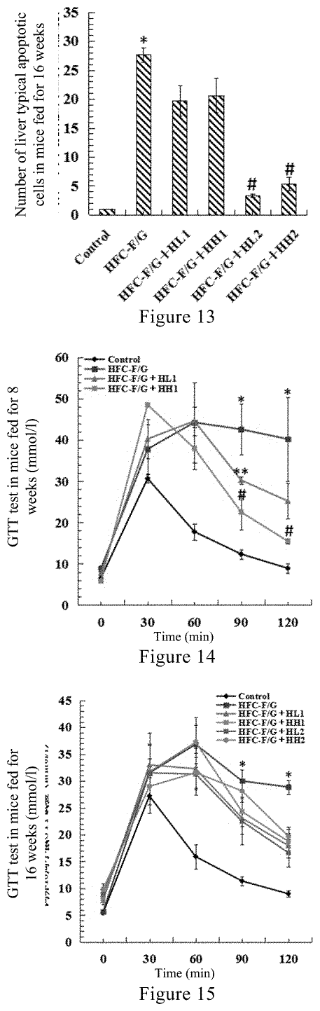

It can be seen from FIG. 13 that at week 16, compared with the normal control group, the number of typical TUNEL staining positive cells in the mouse livers of the model group increased significantly (p<0.01), and the number of apoptotic bodies in the low dose group 2 (HL2) and the high dose group 2 (HH2) was significantly decreased (p<0.01, p<0.01).

4. Glucose Tolerance, Insulin Level and Resistance Index in Mice at Week 8 and Week 16

4.1 Fasting Blood Glucose Level and Glucose Tolerance Test in Mice at Week 8 and Week 16

As can be seen from FIG. 14, compared with the normal control group, the blood glucose level in the model group continued to rise until 60 min and then decreased insignificantly, and there was significant difference at 90 min (p<0.001) and 120 min (p≤0.001). Compared with this, the blood glucose levels in the treatment groups was significantly decreased at 90 min (p<0.05, p<0.01) and 120 min (p<0.01); compared with the normal control group, the blood glucose level in the model group continued to rise until 60 min and then decreased slightly, and there was significant difference at 90 min (p<0.001) and 120 min (p<0.01), and compared with this, the blood glucose in the treatment groups were significantly decreased at 90 min (p<0.05, p≤0.001) and 120 min (p<0.05).

As can be seen from FIG. 15, at week 16, compared with the normal control group, the blood glucose level in the model group continued to rise until 60 min and then decreased slightly, and there was significant difference at 90 min (p≤0.001) and 120 min (p<0.001). Compared with the model group, the blood glucose in the treatment groups had a decreasing trend at 90 min and 120 min; compared with the normal control group, the model control group increased significantly, and there was significant difference at 90 min (p<0.01) and 120 min (p≤0.01). However, compared with the treatment groups, there was a drastically decrease after 60 min and no significant difference was shown.

4.2 Insulin Level and Resistance Index in Mice at Week 16

4.2.1 Fasting Insulin Level in Mice at Week 16

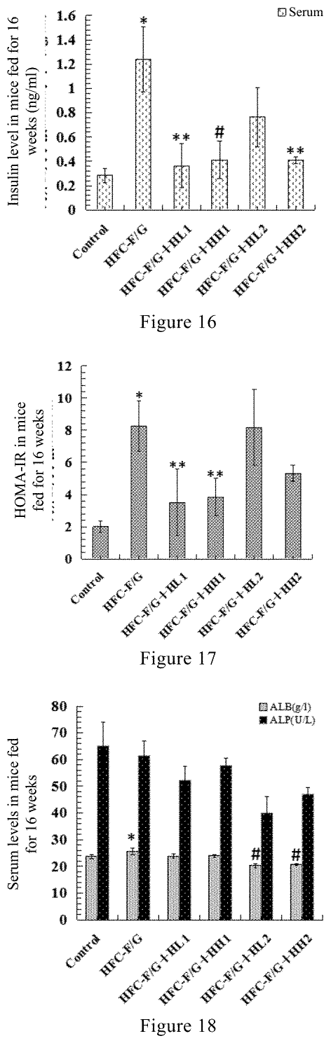

As can be seen from FIG. 16, compared with the normal control group, the insulin level in the model group increased significantly (p<0.001), and the low dose group 1 (HL1), the high dose group 1 (HH1) and the high dose group 2 (HH2) showed significantly decreases (p<0.001, p<0.001, p<0.001) as compared with the model group, and the low dose group 2 (HL2) had a decreasing trend compared with the model group.

4.2.2 Insulin Resistance (HOMA-RI) in Mice at Week 16

As can be seen from FIG. 17, compared with the normal control group, at week 16, the insulin resistance HOMA-RI level in the model group increased significantly (p<0.001), and the low dose group 1 (HL1) and the high dose group 1 (HH1) showed significantly decreases (p<0.05, p<0.05) as compared with the model group, and the high dose group 2 (HH2) had a decreasing trend, while no significant difference was observed for the low dose group 2 (HL2).

5. Serum Biochemical Test at Week 8 and Week 16

5.1 Effect of L47 on Serum ALB and ALP in C57BL6 Mice at Week 16

As can be seen from FIG. 18, at week 16, compared with the normal control group, the ALB level in the model group increased (p<0.05), the low dose group 2 (HL2) and the high dose group 2 (HH2) were slightly decreased (p<0.001, p<0.001), and other groups had no difference. There was no significant difference in the ALP levels among the groups.

5.2 Effect of L47 on Serum HDL-C and LDL-C in C57BL6 Mice at Week 8 and Week 16

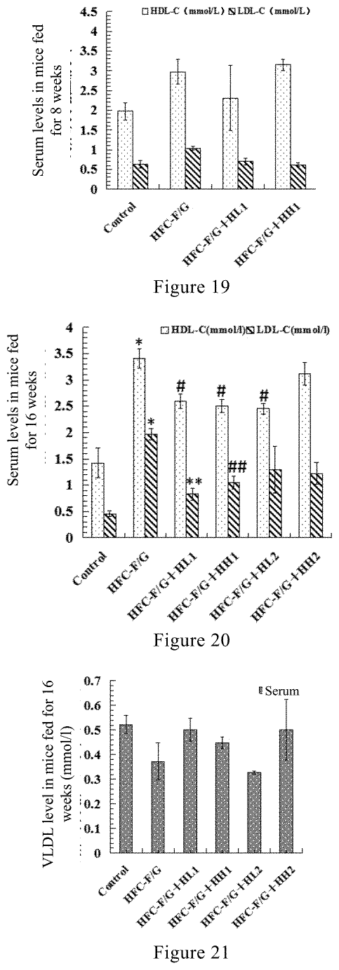

As can be seen from FIG. 19, at week 8, the levels of HDL-C and LDL-C in the model group both increased as compared with the normal control group, the levels of HDL-C and LDL-C in the low dose group 1 (HL1) were both decreased as compared with the model group, and only the high dose group 1 (HH1) had decreased LDL-C levels.

As can be seen from FIG. 20, at week 20, the levels of HDL-C and LDL-C in the model group both increased significantly (p<0.001, p≤0.001) as compared with the normal control group, the HDL-C levels in the treatment groups (except for the high dose group 2 (HH2)) were all decreased compared with the model group (p<0.01), and only the low dose group 1 (HL1) and the high dose group 1 (HH1) had decreased LDL-C levels (p≤0.001, p<0.01).

5.3 Effect of L47 on Serum VLDL in C57BL6 Mice at Week 16

As can be seen from FIG. 21, at week 16, the VLDL level in the model group had a decreasing trend as compared with the normal control group, and levels in other groups (except for the low dose group 2 (HL2)) increased as compared with the model group.

5.4 Effect of L47 on Serum ALT and AST in C57BL6 Mice at Week 8 and Week 16

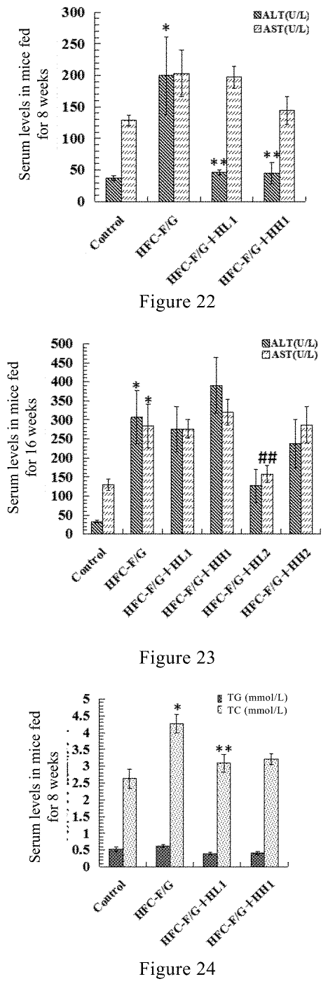

As can be seen from FIG. 22, at week 8, the levels of ALT and AST in the model group both increased as compared with the normal control group, with the ALT levels increased more significantly (p<0.001), and the ALT levels in the treatment groups were significantly decreased ((p≤0.001) compared with the model group. The AST level had a decreasing trend.

As can be seen from FIG. 23, at week 16, the levels of ALT and AST in model group both increased significantly (p<0.01) as compared with the normal control group, the levels of ALT and AST in the low dose group 2 (HL2) were both significantly decreased (p<0.05) compared with the model group, and the levels of ALT and AST in the high dose group 2 (HH2) had decreasing trends, while the levels of ALT and AST in the low dose group 1 (HL1) and the high dose group 1 (HH2) both did not decrease.

5.5 Effect of L47 on Serum TG and TC in C57BL6 Mice at Week 8 and Week 16

As can be seen from FIG. 24, at week 8, the levels of TG and TC in the model group both increased as compared with the normal control group, with the TG levels increased more significantly (p<0.01), and the levels of TG and TC in the treatment groups were all significantly decreased as compared with the model group, wherein the TC levels in the low dose group 1 (HL1) were decreased significantly (p<0.05).

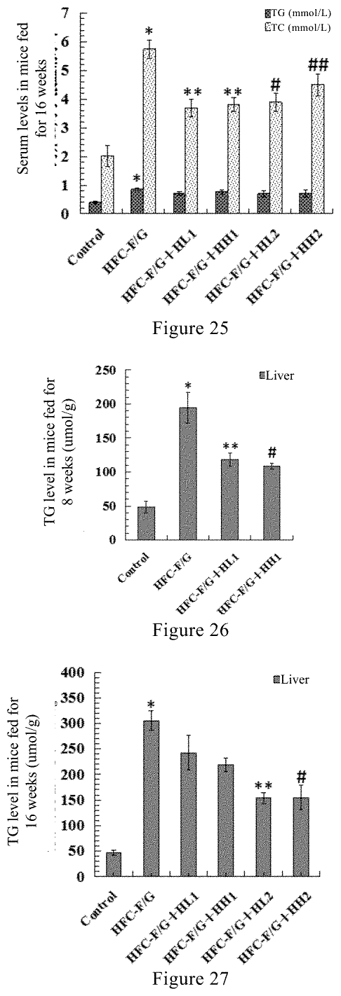

As can be seen from FIG. 25, at week 16, the levels of TG and TC in the model group both increased significantly (p≤0.001, p<0.001) as compared with the normal control group, and TC levels in the treatment groups were significantly decreased (p<0.001, p<0.001, p≤0.001, p<0.05) as compared with the model group, and the TG levels did not decrease.

6. Liver Biochemical Test at Week 8 and Week 16

6.1 Effect of L47 on Liver TG in C57BL6 Mice at Week 8 and Week 16

As can be seen from FIG. 26, at week 8, the TG levels in the model group increased significantly (p<0.001) as compared with the normal control group, and that in the treatment groups were significantly decreased (p≤0.001, p<0.001) as compared with the model group.

As can be seen from FIG. 27, at week 16, the TG levels in the model group increased significantly (p<0.001) as compared with the normal control group, while that in the low dose group 2 (HL2) and the high dose group 2 (HH2) were significantly decreased (p<0.01, p<0.05) as compared with the model group, and the low dose group 1 (HL1) and the high dose group 1 (HH1) had decreasing trends.

6.2 Effect of L47 on Liver TC in C57BL6 Mice at Week 8 and Week 16

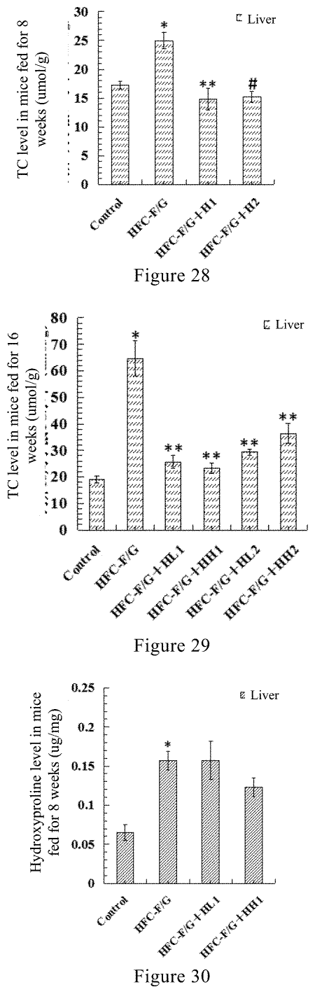

As can be seen from FIG. 28, at week 8, the liver TC levels in the model group increased significantly (p<0.01) as compared with the normal control group, and that in the treatment groups were significantly decreased (p<0.001, p≤0.001) as compared with the model group.

As can be seen from FIG. 29, at week 16, the TC levels in the model group increased significantly (p<0.001) as compared with the normal control group, and that in the treatment groups were significantly decreased (p<0.001, p<0.001, p<0.001, p<0.001) as compared with the model group.

6.3 Effect of L47 on Liver Hydroxyproline in C57BL6 Mice at Week 8 and Week 16

As can be seen from FIG. 30, at week 8, the liver hydroxyproline levels in the model group increased significantly (p<0.001) as compared with the normal control group, and the treatment groups had decreasing trends as compared with the model group.

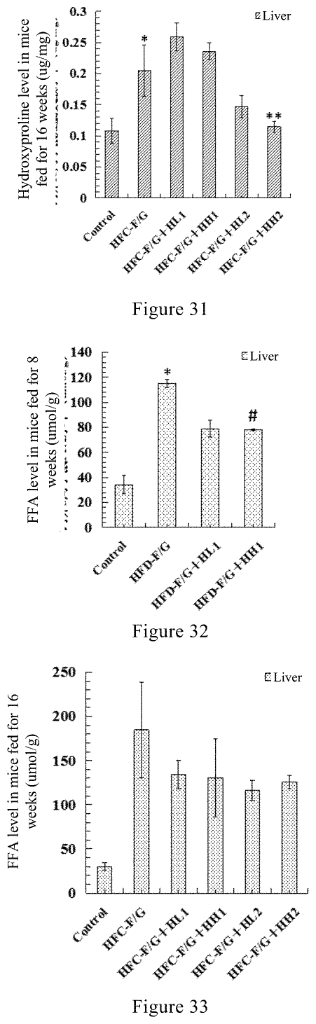

As can be seen from FIG. 31, at week 16, the liver hydroxyproline levels in the model group increased significantly (p<0.01) as compared with the normal control group, while that in the low dose group 2 (HL2) and the high dose group 2 (HH2) were decreased as compare with the model group, wherein HH2 was more significant (p<0.05), and the levels in the low dose group 1 (HL1) and the high dose group 1 (HH1) were higher.

6.4 Effect of L47 on Liver FFA in C57BL6 Mice at Week 8 and Week 16

As can be seen from FIG. 32, at week 8, the liver FFA levels in the model group increased significantly (p<0.001) as compared with the normal control group, and the treatment groups had decreasing trends as compared with the model group (high dose group had statistical significance (p<0.05)).

As can be seen from FIG. 33, at week 16, the liver hydroxyproline level in the model group increased significantly as compared with the normal control group, and the levels in the treatment groups were decreased as compared with the model group.

III. Experimental Conclusions

(I) Effect of L47 on Fat Deposition in C57BL6 Mice

1. Effect of L47 on Body Fat Deposition in C57BL6 Mice

In this experiment, by recording the body weight of mice every week, it is found that the body weights of mice in the L47 treatment groups were significantly decreased compared with the model group and the model control group at week 8. General observation of abdominal fat deposition in mice showed that the treatment groups had significant effect. At week 16, except for the decrease of the low dose group 2, there was no significant difference in body weight between the treatment groups and the model group, and there was no significant difference in abdominal fat deposition among each group. The result of quantitative body fat on CT scan in vivo showed that the subcutaneous fats in the L47 treatment groups decreased significantly as compared with the model group at week 16. In addition, L47 had no significant effect on visceral fat and subcutaneous fat.

2. Effect of L47 on Liver Fat Deposition in C57BL6 Mice

In this experiment, by general observation of liver change, it is found that the livers in the treatment groups are improved compared with the model group, with more normal colors and reduced weights. The levels of liver triglyceride and free fatty acid decreased significantly. The liver HE staining and oil red 0 staining also show that the fat droplets formed in the treatment groups were reduced compared with the model group; at week 16, the treatment effects in the low dose group 2 and the high dose group 2 were more significant compared with the model group, i.e., the liver colors tended to be normal, and the liver were lighter, especially in the low dose group 2. The liver triglyceride levels in the low dose group 2 and the high dose group 2 decreased significantly. The liver free fatty acid levels in the treatment groups had decreasing trends compared with the model group. The liver HE staining and oil red 0 staining also show that the fat droplets formed in the low dose group 2 and the high dose group 2 were less and smaller compared with the model group.

3. Effect of L47 on Serum Fat Deposition in C57BL6 Mice

In this experiment, by measuring serum triglyceride level, it is found that the levels in the treatment groups decreased compared with the model group at week 8, and there was no significant difference between the treatment groups and the model group at week 16.

In conclusion, by the observation of fat deposition in mice at week 8 and week 16, it is found that L47 can effectively reduce the subcutaneous fat, the liver fat and the serum fat deposition in C57BL6 mice in a short period of time and in a dose-dependent manner, but the improvement reduced as the treatment duration extended. L47 had no significant effect on the visceral fat and the subcutaneous fat.

(II) Effect of L47 on Metabolism Level in C57BL6 Mice

In this experiment, through the detection of serum metabolism indexes, it is found that at week 8, the ALB levels and the LDL levels in the treatment groups decreased compared with the model group, and there was no significant difference in the levels of ALP, HDL and VLDL. At week 16, HDL and LDL in the treatment groups decreased compared with the model group, and there was no significant difference in the levels of ALP and VLDL. ALB levels in the low dose group 2 and the high dose group 2 decreased compared with the model group, and there was no significant difference in other indexes;

In addition, through the ELASA detection of fasting serum insulin level, the glucose tolerance test with glucose injected into the abdominal cavity of mice, and further the calculation of insulin resistance index based on the fasting blood glucose level, it is found that at week 16, in addition to the low dose group 2, the insulin levels and the insulin resistance in the treatment groups decreased significantly compared with the model group, and the blood glucoses in the treatment groups decreased compared with the model group.

In summary, through the detection of metabolism indexes in mice at week 8 and week 16, it is found that L47 can reduce the levels of ALB, HDL and LDL in mice, and had no effect on the levels of VLDL and ALP. L47 can effectively reduce blood glucose and insulin level, improve insulin resistance, and the effect was better as dose and administration duration increased.

(III) Effect of L47 on Liver Function in C57BL6 Mice

In this experiment, through the detection of the levels of serum ALT and AST, it is found that the levels of ALT and AST in the treatment groups decreased compared with the model group at week 8. At week 16, the levels of ALT and AST in the low dose group 2 and the high dose group 2 decreased significantly compared with the model group, and the levels of ALT and AST in the high dose group 1 had increasing trends.

In addition, in this experiment, through the TUNEL staining of the liver tissue section, it is found that at week 8, the apoptosis in the treatment groups were improved effectively compared with the model group, and the numbers of typical apoptotic cells also decreased significantly. At week 16, the apoptosis in the low dose group 2 and the high dose group 2 were improved effectively compared with the model group, and the numbers of typical apoptotic cells decreased significantly.

In conclusion, based on the liver function indexes in mice at week 8 and week 16, it is found that L47 can effectively reduce the levels of ALT and AST and improve the apoptosis in C57BL6 mice in a short period of time in a dose-dependent manner.

(IV) Effect of L47 on Liver Fibrosis in C57BL6 Mice

In this experiment, through MASSON staining and the detection of hydroxyproline level in liver tissue, it is found that at week 8, the fiber cords and the hydroxyproline level in the treatment groups were reduced compared with the model group. At week 16, compared with the model group, the low dose group 2 and the high dose group 2 had reduced fiber cords and significantly decreased hydroxyproline levels.

In conclusion, based on the liver fibrosis indexes in mice at week 8 and week 16, it is found that L47 can effectively reduce the fibrosis degree of liver in C57BL6 mice in a short period of time in a dose-dependent manner.

Conclusion: short-term (8 weeks) co-administration or delayed administration of hepalatide (L47) can improve liver fat accumulation, liver cell damage, insulin resistance and liver fibrosis to various degrees. Improvements in insulin resistance persist as the treatment duration extended (16 weeks).

1. A method for treating or preventing nonalcoholic fatty liver disease comprising administering a subject in need thereof a polypeptide comprising an amino acid sequence derived from hepatitis B virus (HBV) or a pharmaceutical composition comprising the polypeptide.

2. The method of claim 1, wherein the nonalcoholic fatty liver disease is selected from simple fatty liver, nonalcoholic steatohepatitis, fatty liver fibrosis and cirrhosis.

3. The method of claim 1, wherein the polypeptide comprises an amino acid sequence of the pre-S1 region of HBV.

4. The method of claim 1, wherein:
one or more amino acid residues of the polypeptide are deleted, substituted, or inserted; and/or
the polypeptide comprises at the N-terminus and/or the C-terminus a native flanking amino acid sequence from the pre-S1 region of HBV.

5. The method of claim 1, wherein the polypeptide comprises the glycine corresponding to amino acid 13 of the pre-S1 region of HBV genotype C, and/or the asparagine corresponding to amino acid 20 of the pre-S1 region of HBV genotype C.

6. The method of claim 1, wherein the polypeptide:
(1) comprises an amino acid sequence selected from SEQ ID NOs: 21-40; or
(2) has at least about 30%, 40%, 50%, 60%, 70%, 80%, 85%, 90%, 95%, 96%, 97%, 98%, or 99%% identity to an amino acid sequence selected from SEQ ID NOs: 21-40.

7. The method of claim 1, wherein:
the polypeptide comprises an N-terminal modification with a hydrophobic group; and/or
the polypeptide comprises a C-terminal modification that is capable of stabilizing the polypeptide.

8. The method of claim 1, wherein the polypeptide comprises SEQ ID NO: 23, and wherein the polypeptide further comprises an N-terminal modification with myristic acid and a C-terminal modification with amination; or wherein the polypeptide comprises SEQ ID NO: 3.

9. The method of claim 1, wherein the pharmaceutical composition further comprises a therapeutically effective amount of at least one second agent.

10. The method of claim 1, wherein the pharmaceutical composition is in a dosage form suitable for administration by at least one mode chosen from parenteral, intrapulmonary, intranasal, intralesional, intramuscular, intravenous, intraarterial, intraperitoneal, and subcutaneous administration.

11. The method of claim 3, wherein the polypeptide comprises an amino acid sequence of the pre-S1 region of HBV genotype A, B, C, D, E, F, G, or H.

12. The method of claim 3, wherein the polypeptide comprises the sequence of amino acids 13-59 of the pre-S1 region of HBV genotype C, or comprises a sequence from the pre-S1 region of HBV genotype A, B, D, E, F, G, or H that corresponds to amino acids 13-59 of the pre-S1 region of HBV genotype C.

13. The method of claim 4, wherein 1-30, 1-20, 1-10, 1-8, 1-5, or 1-3 amino acid residues of the polypeptide are deleted, substituted, or inserted.

14. The method of claim 4, wherein the native flanking amino acid sequence from the pre-S1 region of HBV has 1-10, 1-8, 1-5, or 1-3 amino acids in length.

15. The method of claim 6, wherein the polypeptide comprises the amino acid sequence of SEQ ID NO: 23.

16. The method of claim 7, wherein the hydrophobic group is chosen from myristic acid, palmitic acid, stearic acid, oleic acid, linoleic acid, cholesterol, and arachidonic acid; more preferably, the hydrophobic group is myristic acid.

17. The method of claim 7, wherein the C-terminal modification is amidation or isopentanediolization.

18. The method of claim 9, wherein the second agent is chosen from an antihyperlipidemic agent, an antihyperglycemic agent, an antidiabetic agent, an antiobesity agent, and a bile acid analogue.

19. The method of claim 9, wherein the second agent is chosen from insulin, metformin, sitagliptin, colesevelam, glipizide, simvastatin, atorvastatin, ezetimibe, fenofibrate, nicotinic acid, orlistat, lorcaserin, phentermine, topiramate, obeticholic acid, and ursodeoxycholic acid.

20. The method of claim 2, wherein the polypeptide comprises SEQ ID NO: 23, and wherein the polypeptide further comprises an N-terminal modification with myristic acid and a C-terminal modification with amination; or wherein the polypeptide comprises SEQ ID NO: 3.
